(19) United States
(12) Patent Application Publication
(10) Pub. No.: US 2025/0090836 A1
(43) Pub. Date: Mar. 20, 2025

(54) CIRCULATION SUPPORT DEVICES, SYSTEMS, AND METHODS

(71) Applicant: Boston Scientific Scimed, Inc., Maple Grove, MN (US)

(72) Inventors: Spencer Fodness-Bondhus, Columbia Heights, MN (US); Corydon Carlson, Stillwater, MN (US)

(73) Assignee: Boston Scientific Scimed, Inc., Maple Grove, MN (US)

(21) Appl. No.: 18/889,035

(22) Filed: Sep. 18, 2024

Related U.S. Application Data
(60) Provisional application No. 63/539,242, filed on Sep. 19, 2023.

(51) Int. Cl. A61M 60/546; A61M 60/17; A61M 60/216; A61M 60/585; A61M 60/816

(52) U.S. Cl. CPC A61M 60/546; A61M 60/17; A61M 60/216; A61M 60/585; A61M 60/816; A61M 2205/04; A61M 2205/103; A61M 2205/3327; A61M 2205/3334; A61M 2205/3365; A61M 2205/502; A61M 2210/125; A61M 2230/005; A61M 2230/20; A61M 2230/30

(57) ABSTRACT

A circulatory support system may include a blood pump, one or more sensors, and a controller in communication with the one or more sensors. The blood pump may include a driven component and a motor in communication with the driven component to drive the driven component to pump a blood flow through the blood pump. A sensor of the one or more sensors may be configured to sense a value related to a speed of the motor. The controller may be configured to provide a command signal to the motor to drive the driven component and determine a rate of hemolysis during operation of the blood pump based on an operation parameter of the blood pump.

### CROSS-REFERENCE TO RELATED APPLICATIONS

This application claims the benefit of priority under 35 U.S.C. § 119 of U.S. Provisional Application No. 63/539,242, filed Sep. 19, 2023, the entire disclosure of which is hereby incorporated by reference.

### TECHNICAL FIELD

The present disclosure pertains to mechanical circulatory support devices. More specifically, the present disclosure relates to operation of percutaneous ventricular assist devices (PVADs).

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, cardiac assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including ventricular assist devices.

A first example may include a circulatory support system comprising a blood pump comprising a driven component and a motor in communication with the driven component and configured to drive the driven component to pump a blood flow through the blood pump, one or more sensors configured to sense a measure related to a speed of the motor, and a controller in communication with the motor and the one or more sensors configured to sense the measure related to the speed of the motor, wherein the controller may be configured to send a command signal to the motor and determine a rate of hemolysis during operation of the blood pump based on the speed of the motor.

Alternatively or additionally to any of the examples above, in another example, the controller may be configured to determine a measure related to a flow rate of blood through the blood pump based on the speed of the motor, determine a measure related to a left ventricular pressure of the patient based on the speed of the motor, and determine the rate of hemolysis based on the measure related to the flow rate through the blood pump and the measure related to the left ventricular pressure of the patient.

Alternatively or additionally to any of the examples above, in another example, the controller may be configured to receive a hemolysis test result for a patient including a time at which a sample of blood was taken from the patient and determine the rate of hemolysis based on the speed of the motor, the hemolysis test result, and the time at which the sample of blood was taken from the patient.

Alternatively or additionally to any of the examples above, in another example, the hemolysis test result may include a value of plasma-free hemoglobin in the sample of blood or a value of lactate dehydrogenase (LDH) in the sample of blood.

Alternatively or additionally to any of the examples above, in another example, the controller may be configured to integrate the rate of hemolysis determined during a period of operation of the blood pump to determine an amount of hemolysis that has occurred during the period of operation.

Alternatively or additionally to any of the examples above, in another example, the controller may be configured to automatically adjust the command signal based on the rate of hemolysis determined during operation of the blood pump.

Alternatively or additionally to any of the examples above, in another example, the controller may be configured to automatically adjust the command signal to reduce a flow rate of blood through the blood pump based on the rate of hemolysis determined during operation of the blood pump.

Alternatively or additionally to any of the examples above, in another example, the controller may be configured to determine an amount of hemolysis based on the rate of hemolysis determined during operation of the pump, and output a signal indicating a hemolysis test is recommended when the amount of hemolysis has reached or gone beyond a threshold level.

Alternatively or additionally to any of the examples above, in another example, the controller may be configured to receive an input from a user, determine an expected rate of hemolysis during operation of the blood pump based on the input received from the user, and output an indication that a blood pump change is recommended based on the expected rate of hemolysis determined being above a threshold.

Alternatively or additionally to any of the examples above, in another example, the controller may be configured to output an indication to a user interface based on the rate of hemolysis determined during operation of the blood pump.

A further example may include a non-transitory computer readable medium having stored thereon instructions executable by a circulatory support device for use with a heart of a patient, the instructions may cause the circulatory support device to perform a method comprising sending a command signal from a controller to a motor of a blood pump to cause the motor of the blood pump to drive a driven component to pump blood from a ventricle of the heart of the patient through the blood pump to vasculature of the patient, receiving a measure related to a speed of the motor from one or more sensors in communication with the controller, and determining a rate of hemolysis during operation of the blood pump based on the speed of the motor.

Alternatively or additionally to any of the examples above, in another example, determining the rate of hemolysis during operation of the blood pump further comprises determining a flow rate of blood through the blood pump based on the speed of the motor, determining a measure related to a ventricular pressure of the patient based on the speed of the motor, and determining the rate of hemolysis based on the measure related to the flow rate and the measure related to the ventricular pressure of the patient.

Alternatively or additionally to any of the examples above, in another example, the method may further comprise receiving a hemolysis test result at a specified time at which a tested blood sample was taken, wherein the rate of hemolysis during operation of the blood pump may be determined based on the hemolysis test result and the specified time.

Alternatively or additionally to any of the examples above, in another example, the method may further comprise integrating the rate of hemolysis determined during a period of operation of the blood pump to determine an amount of hemolysis during the period of operation of the blood pump.

Alternatively or additionally to any of the examples above, in another example, the method may further comprise outputting a signal indicating a hemolysis test is recommended when the amount of hemolysis has reached or gone beyond a threshold level.

Alternatively or additionally to any of the examples above, in another example, the method may further comprise automatically adjusting the command signal based on the rate of hemolysis determined during operation of the blood pump.

A further example may include a method of operating a blood circulatory support system for use with a heart of a patient, the method comprising sending a command signal from a controller to a motor of a blood pump to cause the motor of the blood pump to drive a driven component to pump blood from a ventricle of the heart of the patient through the blood pump to vasculature of the patient, receiving a measure related to a speed of the motor from one or more sensors in communication with the controller, and determining a rate of hemolysis during operation of the blood pump based on the speed of the motor.

Alternatively or additionally to any of the examples above, in another example, determining the rate of hemolysis during operation of the blood pump may further comprise determining a flow rate of blood through the blood pump based on the speed of the motor, determining a measure related to a ventricular pressure of the patient based on the speed of the motor, and determining the rate of hemolysis based on the measure related to the flow rate and the measure related to the ventricular pressure of the patient.

Alternatively or additionally to any of the examples above, in another example, the method may further comprise determining an amount of hemolysis during a period of operation of the blood pump based on the rate of hemolysis determined during the period of operation of the blood pump and outputting a signal indicating a hemolysis test is recommended when the amount of hemolysis has reached or gone beyond a threshold level.

Alternatively or additionally to any of the examples above, in another example, the method may further comprise automatically adjusting the command signal based on the rate of hemolysis determined during operation of the blood pump.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic partial cross-section of anatomy and a schematic side view of an illustrative percutaneous ventricular assist device (PVAD) within the anatomy;

FIG. 2 is a schematic cross-sectional view of an illustrative PVAD;

FIG. 3 is a schematic detailed view of the illustrative PVAD depicted in FIG. 2, taken within line 3-3;

FIG. 4 is a schematic diagram of an illustrative circulatory support system;

FIG. 5 is a schematic diagram of an illustrative computing device or controller and user interface;

FIG. 6 is a schematic diagram of an illustrative circulatory support system;

FIG. 7 is a schematic diagram of an illustrative circulatory support system;

FIG. 8 is a schematic diagram of an illustrative controller configuration;

FIG. 9 is a schematic diagram of an illustrative controller configuration;

FIG. 10 is a schematic diagram of an illustrative controller configuration;

FIG. 11 is a schematic diagram of an illustrative controller configuration;

FIG. 12 is a schematic diagram of an illustrative controller configuration;

FIG. 13 is a schematic diagram of an illustrative controller configuration; and

FIG. 14 is a schematic diagram of an illustrative method of operating a circulatory support system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist devices are known for assisting or replacing a pumping function of a heart in a patient with severe heart failure and/or other cardiac conditions. Circulatory assist devices may be configured to treat patients with cardiogenic shock, myocardial infraction, acutely decompensated heart failure, and/or other heart related conditions. Additionally or alternatively circulatory assist devices may support a patient during percutaneous coronary interventions and/or other procedures.

Example cardiac circulatory assist devices include, but are not limited to, ventricular assist devices (VADs), total artificial hearts, intra-aortic balloon pumps (IABP), and extracorporeal membrane oxygenation (ECMO). Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. A PVAD may be placed at a desired location of anatomy of a patient via percutaneous access and delivery, which may enable the PVAD to be used in emergency medicine, a cath lab, and/or other surgical and/or non-surgical settings.

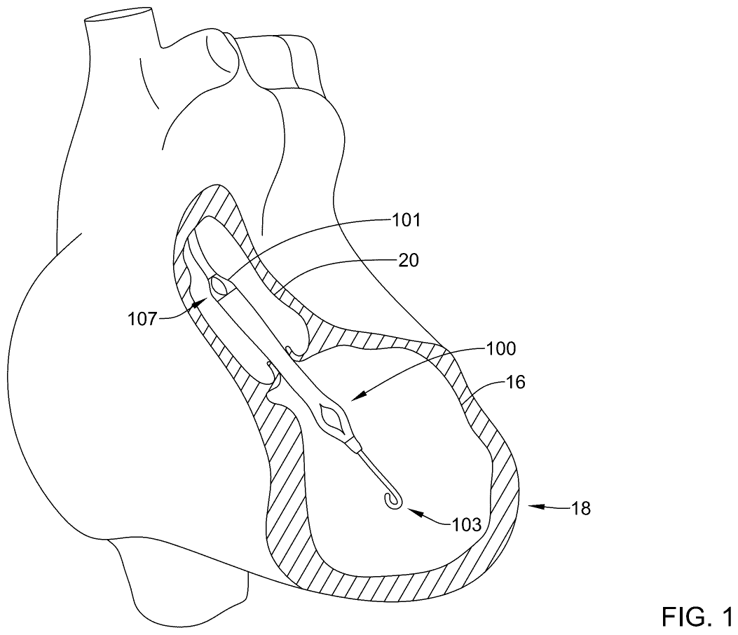

FIG. 1 depicts an illustrative positioning of a blood pump 100 (e.g., a percutaneous circulatory support device, such as a PVAD in an LVAD configuration, etc.) in anatomy of a patient. In FIG. 1, the blood pump 100 is positioned with a distal end 103 located in a left ventricle 16 of a heart 18 and a proximal end 107 in an aorta 20, such that the blood pump 100 extends across an aortic valve 22 between the left ventricle 16 and the aorta 20. With the blood pump 100 extending from the left ventricle 16 to the aorta 20, the blood pump 100 may be configured to pump blood from the left ventricle 16 into the aorta 20 to assist blood flow circulation. Other suitable positions of the blood pump relative to the anatomy are contemplated and include, but are not limited to, the distal end 103 of the blood pump being positioned in a right ventricle of the heart 18 with a proximal end being positioned in a pulmonary artery.

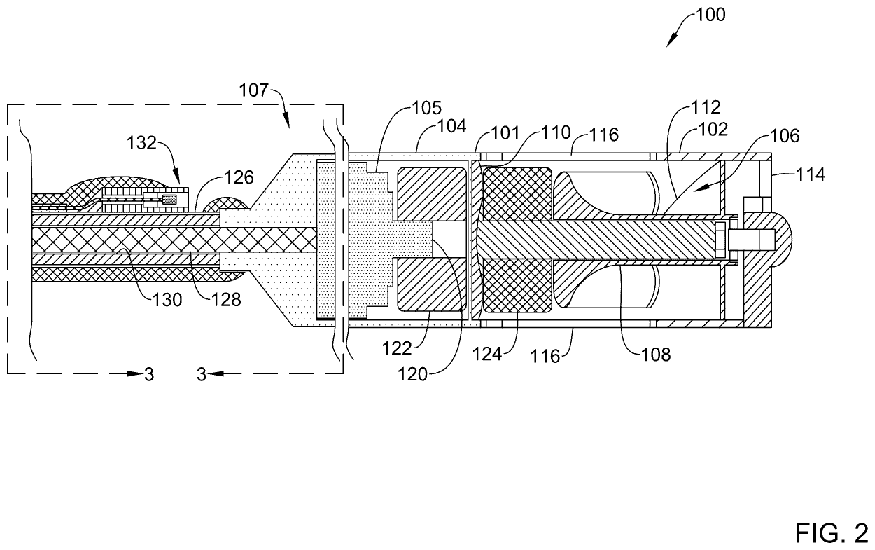

FIG. 2 depicts a schematic cross-sectional view of an illustrative configuration of the blood pump 100. In some cases, the blood pump 100 may form part of a percutaneous circulatory or circulation support system, together with a guidewire, an introducer sheath, a controller, a user interface, one or more sensors, and/or other suitable components.

The blood pump 100 may include a housing 101 having an impeller housing 102 and a motor housing 104. The impeller housing 102 and the motor housing 104 may be integrally or monolithically constructed, but this is not required, and the impeller housing 102 and the motor housing 104 may be separate components configured to be removably or permanently coupled. In some configurations, the blood pump 100 may lack a motor housing 104 separate from the impeller housing 102, and the impeller housing 102 may be coupled directly to a motor 105, or the motor housing 104 may be integrally constructed with the motor 105.

The impeller housing 102 may house an impeller assembly 106 and a driven magnet 124, which may be part of or separate from the impeller assembly 106. The impeller assembly 106 may include an impeller shaft 108 that is rotatably supported by at least one bearing, such as a bearing 110 and/or other suitable bearings. The impeller assembly 106 may further include an impeller 112 that rotates relative to the impeller housing 102 to drive blood through the blood pump 100. In some configurations and, for example as illustrated, the impeller shaft 108 and the impeller 112 may be separate components, and in other configurations the impeller shaft 108 and the impeller 112 may be integrated. The impeller assembly 106, as a whole, may be considered a driven component and/or the rotating components of the impeller assembly 106 (e.g., the impeller shaft 108 and/or the impeller 112) may individually or in combination be driven components.

The impeller 112 may be configured within the impeller housing 102 such that as the impeller 112 rotates, blood flows from a blood inlet 114 formed on or at the impeller housing 102, through the impeller housing 102, and out of a blood outlet 116 formed on or at the impeller housing 102. In some configurations, the impeller housing 102 may couple to or include a distally extending cannula (not shown), and the cannula may receive and deliver blood to the inlet 114 (e.g., from the left ventricle 16 of the heart 18 and/or from other suitable locations).

The inlet 114 and the outlet 116 may each have any suitable number of apertures configured to facilitate receiving blood at the blood pump 100 and outputting blood from the blood pump 100, respectively. In some examples, the inlet 114 and/or the outlet 116 may each include multiple apertures and in other examples, one or both of the inlet 114 and the outlet 116 may each include a single aperture.

The inlet and the outlet 116 may each be formed at any suitable location along the impeller housing 102 or other suitable location along the blood pump 100. In some examples, and as depicted in FIG. 2, the inlet 114 may be formed on an end portion (e.g., a distal end portion) of the impeller housing 102 and the outlet 116 may be formed on a side portion (e.g., proximal of the location of the inlet 114) of the impeller housing 102. Other suitable positioning configurations of the inlet 114 and/or the outlet 116 on the impeller housing 102 are contemplated.

The motor housing 104 may house the motor 105, along with other suitable components. In some examples, and as depicted in FIG. 2, the motor housing 104 may house at least the motor 105, the drive shaft 120, and a driving magnet 122.

The motor 105 may be any suitable type of motor. In one example, the motor 105 may be a brushless direct current (DC) motor (BLDC), but other suitable motor types are contemplated.

In operation, the motor 105 may be configured to rotatably drive the impeller 112 relative to the impeller housing 102. In some example configurations, the motor 105 may rotate a drive shaft 120, which is coupled to a driving magnet 122. Rotation of the driving magnet 122 may cause rotation of the driven magnet 124, which is part of or connected to and rotates together with the impeller assembly 106. That is, when the impeller shaft 108 is included in the impeller assembly 106, the impeller shaft 108 and the impeller 112 are configured to rotate with the driven magnet 124. Additionally or alternatively, the motor 105 may couple to the impeller assembly 106 via other components.

As discussed in greater detail below, a controller (not shown in FIG. 2) may be operably coupled to the motor 105 and configured to control the motor 105 via one or more command signals sent from the controller to the motor 105. The controller may be disposed within the motor housing 104 and/or the controller may be disposed outside of the motor housing 104 (e.g., in a housing of the blood pump 100 independent from the motor housing 104, exterior of the patient, etc.). In some embodiments, the controller may include multiple components, one or more of which may be disposed within and/or separately from the motor housing 104.

The motor housing 104 may couple to a catheter 126 at a location of the motor housing 104 opposite the impeller housing 102. The catheter 126 may couple to the motor housing 104 in various manners, such as laser welding, soldering, or the like. The catheter 126 may extend proximally away from the motor housing 104.

The catheter 126 may include one or more lumens for receiving one or more components of a circulatory support system including the blood pump 100. In some cases, the catheter 126 may be configured to carry a motor cable 128 (e.g., one or more cables configured to facilitate operation of the motor 105) within a main lumen 130, and the motor cable 128 may operably couple the motor 105 to the controller (not shown) and/or an external power source (not shown).

The catheter 126 may carry a sensor assembly 132 for measuring pressure within the vasculature of a patient, for example, within the aorta or pulmonary artery. The sensor assembly 132 may be positioned, relative to the other components of the blood pump 100, in a location for obtaining highly accurate pressure data. For example, the proximal position of the sensor assembly 132 relative to the motor housing 104 and the motor 105 may reduce and/or eliminate the motor speed-related or dynamic pressure-related sensing inaccuracies. Such inaccuracies are typical of other percutaneous circulatory support devices that employ pressure sensors located more distally relative to the motor or impeller assembly, for example, devices that employ pressure sensors located near the outlet 116.

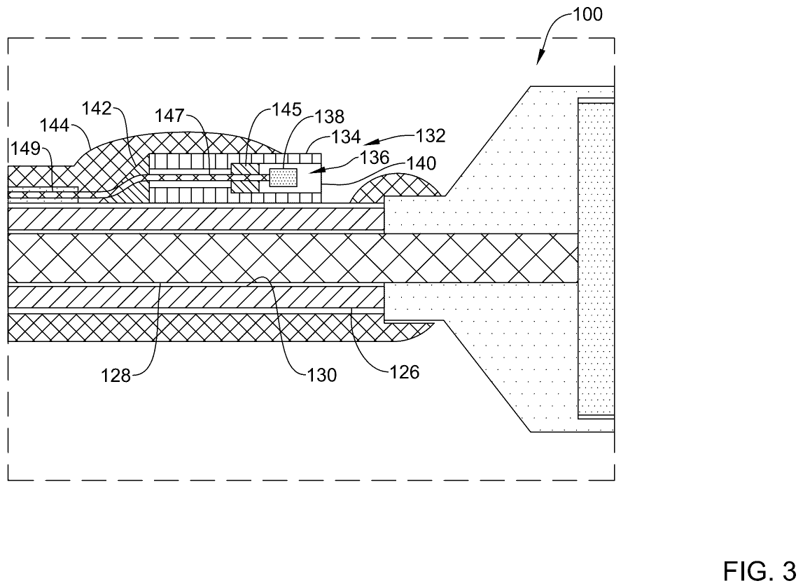

FIG. 3 depicts a schematic detailed view of an interior of line 3-3 in FIG. 2. As depicted in in FIG. 3, the sensor assembly 132 may include a sensor housing 134 having an internal chamber 136. In some examples, the internal chamber 136 may have a counterbore-shape, but other suitable shapes and/or configurations of the internal chamber 136 are contemplated. A pressure sensor 138, such as an optical pressure sensor (e.g., an optical pressure sensor using one or more fiber optics and/or other suitable optical pressure sensor), an electrical pressure sensor, and/or other suitable pressure sensors, may be disposed within the internal chamber 136 and configured to sense a pressure in the aorta 20 when the blood pump 100 extends into the left ventricle 16 of the heart 18. The sensor housing 134 may protect the pressure sensor 138 during deployment of the blood pump 100. The sensor housing 134 may also include a distally facing aperture 140 of or coupled to the internal chamber 136. The aperture 140 may permit blood to enter the internal chamber 136, and the aperture 140 thereby permits the pressure sensor 138 to sense the pressure of the blood proximate the internal chamber 136.

The sensor housing 134 may take various forms. For example, the sensor housing 134 may be a tube or ferrule manufactured from, for example, one or more metals, one or more plastics, composites, and/or other suitable materials. The sensor housing 134 may be coupled to the catheter 126 via one or more weldments (not shown), one or more adhesives 142, and/or an outer jacket 144 surrounding at least a portion of the sensor housing 134 and the catheter 126. The sensor housing 134 may also include a sensor mount 145 within the internal chamber 136. The sensor mount 145 may facilitate supporting the pressure sensor 138 apart from the walls of the sensor housing 134 (e.g., the sensor mount 145 may center the pressure sensor 138 within the internal chamber 136), which in turn facilitates high-accuracy pressure sensing. Other suitable configurations of the sensor housing 134 are contemplated.

The sensor assembly 132 may include a sensor cable 147 coupled to the pressure sensor 138. The sensor cable 147 may operably couple the pressure sensor 138 to the controller (not shown). As illustrated, the sensor cable 147 may extend through the sensor mount 145 and support the pressure sensor 138 apart from the walls of the sensor housing 134. The sensor cable 147 may extend proximally, through the adhesive 142, and through a cable lumen 149 of or coupled to the catheter 126. In some examples, the cable lumen 149 may be coupled to the catheter 126 via one or more weldments (not shown), an adhesive (not shown), and/or the outer jacket 144. In other examples, the cable lumen 149 may be omitted, and the sensor cable 147 may extend through the main lumen 130 of the catheter 126 or lie directly under the outer jacket 144. Example suitable sensor assemblies 132 are disclosed in U.S. Patent Application Publication No. 2023/0149699 A1, filed on Nov. 16, 2022, and titled PERCUTANEOUS CIRCULATORY SUPPORT DEVICE INCLUDING PROXIMAL PRESSURE SENSOR, which is hereby incorporated by reference in its entirety.

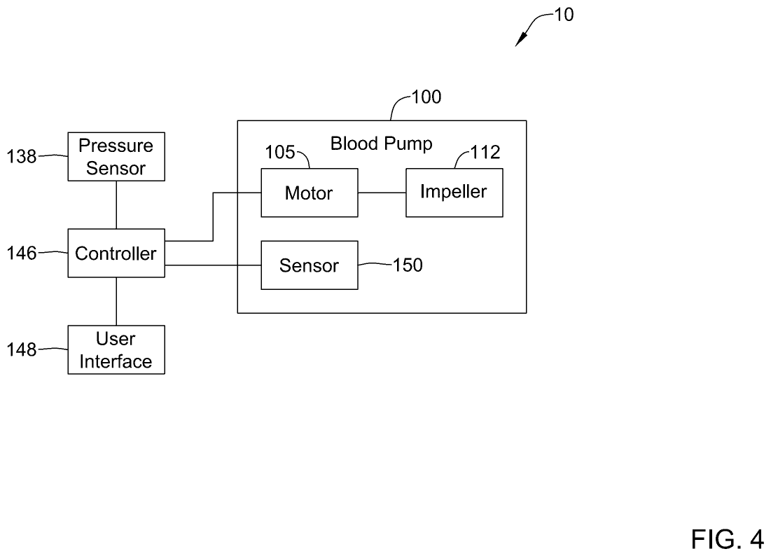

FIG. 4 depicts a schematic diagram of an illustrative circulatory support system 10. Among other additional and/or alternative components, the circulatory support system 10 may include the blood pump 100, the pressure sensor 138, a controller 146, and a user interface 148. As discussed, the blood pump 100 may include a motor 105 in communication with the controller 146 and the impeller 112 in communication with the motor 105.

In some examples, the blood pump 100 may include or be coupled to one or more sensors 150 (e.g., one or more position sensors and/or other suitable speed sensors) configured to sense a speed of the motor 105 and/or a measure related to a speed of the motor 105. When one or more of the sensors 150 are included, the one or more sensors 150 may be coupled to the controller 146 via one or more cables extending through and/or along the catheter 126. In some cases, the speed or position of the motor 105 may be sensed directly from the electrical signals used to drive the motor 105 by the controller 146. In such cases, the motor 105 may be an implicit sensor used with or obviating the need for an explicit sensor 150.

The one or more sensors 150 may be any suitable type(s) of sensor for sensing a speed of a motor 105. Example suitable type(s) of sensors 150 include, but are not limited to, position sensors, Hall effect sensors, magnetic inductive sensors, optical encoders, eddy current sensors, doppler effect sensors, tachometers, and/or other suitable types of sensors.

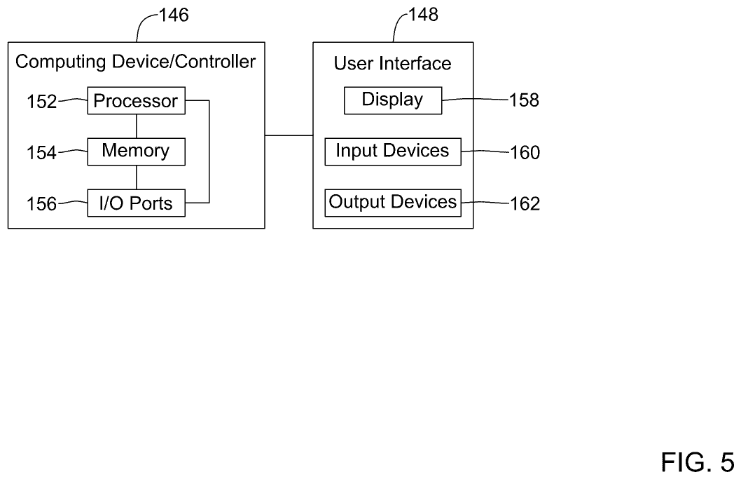

FIG. 5 depicts a schematic diagram of an illustrative configuration of the controller 146 (e.g., computing device) and the user interface 148 of the circulatory support system 10. The controller 146 may be and/or may include any suitable computing device configured to process data of or for the circulatory support system 10 (e.g., of or from the motor 105, the pressure sensor 138, the sensor 150, patient test results, user inputs, patient monitors, etc.) In some cases, one or more components of the circulatory support system 10 may be incorporated into the controller 146 and/or the user interface 148. Further, one or more components of the circulatory support system 10 may incorporate one or more computing devices similar to or having components similar to the controller 146 and/or the user interface 148.

The controller 146 may be configured to facilitate operation of the circulatory support system 10. The controller 146, in some cases, may be configured to control operation of the motor 105, the pressure sensor 138, the user interface 148, and/or the sensor 150 by establishing and/or outputting control signals to components of the motor 105, the pressure sensor 138, the user interface 148, and/or the sensor 150 to control and/or monitor operation of these units and devices.

The controller 146 may communicate with a remote server or other suitable computing device. When the controller 146, or at least a part of the controller 146, is a component separate from a structure of the motor 105, the pressure sensor 138, the user interface 148, and/or the sensor 150, the controller 146 may communicate with electronic components of the circulatory support system 10 over one or more wired or wireless connections or networks (e.g., LANs and/or WANs).

The controller 146 may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller 146 may be referred to herein in the singular, the controller 146 may be implemented in multiple instances, distributed across multiple computing devices, instantiated within multiple virtual machines, and/or the like.

The illustrative controller 146 may include, among other suitable components, one or more processors 152, memory 154, and/or one or more I/O units 156. Example other suitable components of the controller 146 that are not specifically depicted in FIG. 2 may include, but are not limited to, communication components, a touch screen, selectable buttons, a housing, and/or other suitable components of a controller. As discussed above, one or more components of the controller 146 may be separate from the components of the circulatory support system 10 and/or incorporated into the components of the circulatory support system 10.

The controller 146 may include and/or be in communication with a variety of sub-controllers. Example sub-controllers that may be included in or in communication with the controller 146 may include, but is not limited to, a motor sub-controller, a flow rate sub-controller, a pressure sub-controller, a motor torque sub-controller, a motor mechanical loss sub-controller, a stall pressure sub-controller, a pressure loss sub-controller, and/or other suitable sub-controllers.

The processor 152 of the controller 146 may include a single processor or more than one processor working individually or with one another. The processor 152 may be configured to receive and execute instructions, including instructions that may be loaded into the memory 154 and/or other suitable memory. Example components of the processor 152 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 154 of the controller 146 may include a single memory component or more than one memory component each working individually or with one another. Example types of memory 154 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 154 may be or may include a non-transitory computer readable medium. The memory 154 may include instructions stored in transitory and/or non-transitory state on a computer readable medium that may be executable by the processor 152 to cause the processor to perform one or more of the methods and/or techniques described herein.

The I/O units 156 of the controller 146 may include a single I/O component or more than one I/O component each working individually or with one another. Example I/O units 156 may be or may include any suitable types of communication hardware and/or software including, but not limited to, communication ports configured to communicate with electronic components of the circulatory support system 10 and/or with other suitable computing devices or systems. Example types of I/O units 156 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 156.

The user interface 148 may be configured to communicate with the controller 146 via one or more wired or wireless connections. In some cases, the user interface 148 may be configured to receive sensed and/or calculated or determined data from the controller 146. The user interface 148 may include one or more display devices 158, one or more input devices 160, one or more output devices 162, and/or one or more other suitable features.

The display device 158 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 160 may be and/or may include any suitable components and/or features for receiving user input via the user interface. Example input device(s) 160 include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s) 162 may be and/or may include any suitable components and/or features for providing information and/or data to users and/or other computing components. Example output device(s) 162 include, but are not limited to, displays, speakers, vibration systems, tactile feedback systems, optical outputs, cables, lights, and/or other suitable output devices.

Mechanical circulatory support (MCS) devices, such as a PVAD, can provide transient support for up to approximately several weeks in patients with compromised heart function and/or cardiac output. Operation of such MCS devices, however, may cause some amount of hemolysis due to the nature of the interaction between the MCS device and the blood passing through the MCS device. Hemolysis is a rupturing of red blood cells and the release of their contents into surrounding fluid.

Hemolysis is a concern with MCS devices on the market due to a significant rate of adverse effects from hemolysis. Example adverse effects in a patient from hemolysis include, but are not limited to, production of thrombi, fatigue, dizziness, pale skin, heart palpitations (tachycardia), shortness of breath, yellowing of skin, yellowing of the sclera, enlarged spleen, and/or other adverse effects.

An amount of hemolysis is proportional to a level of support provided by the MCS device (e.g., flow rate may be proportional to hemolysis). As such, physicians may lower the blood flow support of the MCS device to reduce hemolysis caused by the MCS device. However, management of hemolysis caused by operation of the MCS device may be difficult and may not be initiated until after a problem or adverse effect from hemolysis is identified. The concepts discussed herein improve the operation of MCS devices and improve the application of MCS devices to patients due to providing proactive control techniques for operating MCS devices to address a potential for hemolysis caused by operation of the MCS device prior to an occurrence of adverse effects and to mitigate hemolysis and/or adverse effects caused by hemolysis due to operation of the MCS device.

Various operational parameters (e.g., motor speed, pressure, flow, etc.) of, through, or proximate the blood pump 100 may be operationally and/or clinically relevant performance parameters for controlling operation of the blood pump 100 to address hemolysis. For example, users (e.g., clinicians, physicians, etc.) may be interested in having circulatory parameter data, ventricular pressure data, vasculature pressure data (e.g., aortic pressure data, pulmonary artery pressure data, etc.), differential pressure data across the blood pump 100 (e.g., between the ventricle and aorta, etc.), flow data related to blood flowing through the blood pump 100, hemolysis test result data, and/or other data related to operation of the blood pump 100 for making operational decisions to address hemolysis related to the operation of the blood pump 100. Further, pressure and flow data related to and/or for blood flowing through the blood pump 100, other blood pump data related to operation of the blood pump 100, and/or calculations based on data related to blood flowing through the blood pump 100 may be utilized to control operation of the blood pump 100 (e.g., adjust command signals, etc.) automatically by the controller 146 and/or in response to user input to address hemolysis issues.

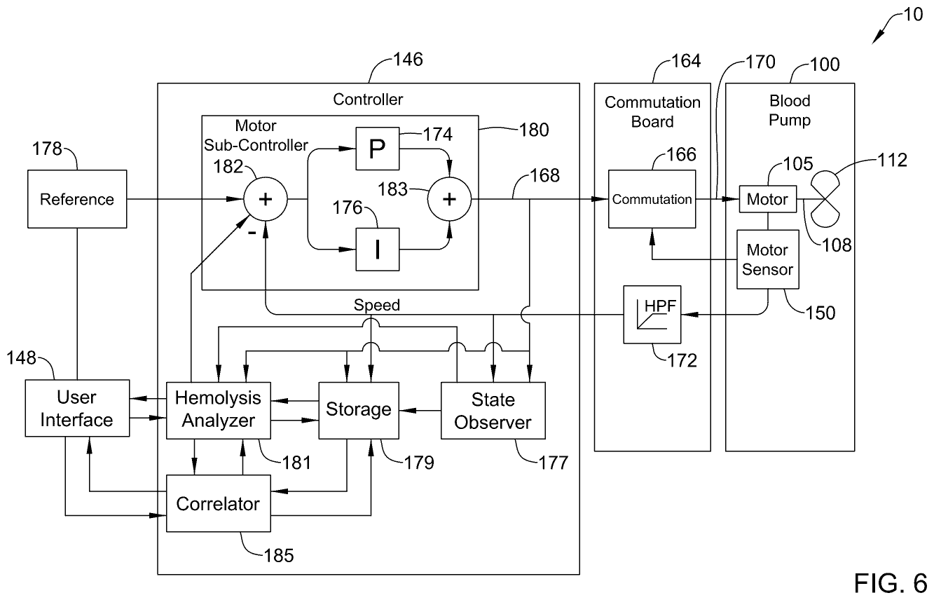

FIG. 6 depicts a schematic diagram of a control system of the circulatory support system 10. As depicted in FIG. 6, the controller 146 may be in communication with the motor 105 of the blood pump 100 via a commutation board 164 to drive or otherwise rotate the impeller shaft 108 and the impeller 112. The commutation board 164 and/or components thereof may be incorporated into the controller 146, may be incorporated into the motor 105, and/or may be a separate component from one or both of the controller 146 and the motor 105. In some examples, the commutation board 164 may be omitted.

In operation, a commutation block 166 of the commutation board 164 may be configured to receive a command signal 168 and an output of the sensor 150 sensing the speed of and/or a measure related to the speed of the motor 105. The commutation block 166 may synchronize the command signal 168 with the operation of the motor 105 using the output of the sensor 150 and provide a control signal 170 to the motor 105 based on the command signal 168 and the output of the sensor 150.

Further, a high pass filter (HPF) 172 of the commutation board 164 may be configured to receive the output of the sensor 150. The HPF 172 may be configured to take the derivative of the position signal and filter noise in the resulting speed signal from the sensor 150 and provide a filtered signal as output to the controller 146. In some examples, the HPF 172 may be omitted, the HPF may be replaced with a LPF (Low Pass Filter) if a speed sensor is used, and/or other suitable filters may be utilized.

The controller 146 may be and/or may include any suitable type of controller. For example, the controller 146 may be and/or include one or more proportional controllers, proportional integral (PI) controllers, proportional integral derivative (PID) controllers, lead lag controllers, non-linear table controllers, linear table controllers, and/or other suitable types of controllers. In some examples, the controller 146 may be or may include one or more PI controllers having a proportional component 174 and an integral component 176, as depicted in FIG. 6. Further, although not required, the controller 146 may include multiple control loops and/or may be configured to regulate intermediate states.

In operation, the controller 146 may be configured to receive a value of the reference parameter 178 that is input from the user interface 148 or automatically produced by the controller 146 to a motor sub-controller 180. The motor sub-controller 180 may include the proportional component 174 and the integral component 176 of a PI controller that are configured to process the value of the reference parameter 178, a value related to the value of the reference parameter 178, and/or other suitable data and output the command signal 168.

The value of the reference parameter 178 may be any suitable type of input from a user, component, or system in communication with the controller 146. In some cases, the value of the reference parameter 178 may be a set point, hemolysis test result, etc. provided via the user interface 148 by a user, but this is not required. The value of the reference parameter 178 may be a set point related to any parameter relevant to operation of the blood pump 100 including, but not limited to, a motor speed, a flow rate of blood across the blood pump 100, a pressure in a ventricle of the heart 18 (e.g., left ventricular pressure and/or right ventricular pressure), a differential pressure across the blood pump 100 (e.g., a difference in pressure between a pressure in a ventricle and a pressure in an aorta), and/or other suitable value. In some examples, the value of the reference parameter 178 may be a speed of the motor 105, but this is not required.

When the value of the reference parameter 178 is a set point for a speed of the motor 105, the value of the reference parameter 178 may be configured to be summed with the output from the sensor 150 (e.g., which may or may not be passed through the HPF 172) and/or an output of a hemolysis analyzer 181 at a summer 182 (e.g., a summation component). When the value of the reference parameter 178 is not a set point for a speed of the motor 105, a value of a parameter based on the value of the reference parameter 178 (e.g., a speed determined based on the value of the reference parameter 178) may be summed with the output of the sensor 150 and/or the hemolysis analyzer 181 at the summer 182. Alternatively or additionally, when the value of the reference parameter 178 is not a set point for a speed of the motor 105, the value of the reference parameter 178 may be summed with a value of a parameter based on the output of the sensor 150 (e.g. pressure, flow, etc.) and/or the output of the hemolysis analyzer 181. In some examples, a difference between the values may be identified at the summer 182 and/or other summers discussed herein, which may be represented by the “-” sign proximate the summer.

Although a PI controller is depicted in FIG. 6 as being the type of controller between the summer 182 and the summer 183, additional and/or alternative controller types may be utilized. When the proportional component 174 and the integral component 176 of the controller 146 are included in the motor sub-controller 180, the output of the summer 182 may be processed with the proportional component 174 and the integral component 176. The outputs of the proportional component 174 and the integral component 176 may be summed with one another at a summer 183, which may output the command signal 168. The values received at the summer 183 may be summed to produce the command signal 168, but this is not required. Other suitable configurations of the motor sub-controller 180 are contemplated.

The controller 146 may utilize outputs and/or signals from the control and/or operation of the blood pump 100 including the motor 105 and the impeller 112 (e.g., the command signal 168, the sensed motor speed, a sensed aortic pressure, etc.) to determine or calculate one or more parameters. Additionally or alternatively, the controller 146 may utilize an output of one or more other sensors of or in communication with the blood pump 100 or the circulatory support system 10 to determine or calculate the one or more parameters, where the output from the one or more sensors may include, but are not limited to, output from the pressure sensor 138 configured to sense a pressure in the vasculature of the patient (e.g., the aorta 20, the pulmonary artery, etc.)

The parameters determined or calculated by the controller 146 may include one or more values of parameters related to blood flow pumped through the blood pump 100. In some examples, the controller 146 may be configured to determine or calculate one or more values of a flow rate of blood across the blood pump 100, one or more pressures proximate the blood pump 100 (e.g., a left ventricular pressure, a right ventricular pressure, a differential pressure across the blood pump 100, etc.), one or more measures of or related to hemolysis caused by operation of the blood pump 100, and/or other suitable values of parameters related to blood flow pumped through the blood pump 100.

To facilitate determining or calculating one or more values of parameters related to blood flow pumped through the blood pump 100 and/or otherwise related to operation of the blood pump 100, the controller 146 may include a state observer 177. The state observer 177 may be configured to receive outputs and/or signals from the controller 146 and/or operation of the motor 105 and the impeller 112 (e.g., the command signal 168, the sensed motor speed, sensed pressure, sensed flow rates, etc.). In some examples and as depicted in FIG. 6, the state observer 177 may be configured to receive the command signal 168 and a sensed motor speed from the motor speed sensor 150. Other configurations are contemplated and additional or alternative inputs may be received at the state observer 177.

The state observer 177 may be configured to monitor the inputs received thereat and determine or calculate values of one or more parameters related to the operation of the blood pump 100 based on the inputs. For example, the state observer 177 may be configured to use received values of parameters (e.g., values of the command signal 168, values related to a speed of the motor 105, and/or other suitable values) to calculate or determine a flow rate of blood through the blood pump 100, pressure provided by the blood pump 100, ventricular pressure, differential pressure across the blood pump 100, arterial pressure (e.g., mean arterial pressure (MAP)), a stall pressure of the motor 105, a theoretical head pressure for the blood pump 100, a pressure loss across the blood pump 100, motor mechanical loss, motor torque, and/or values of other parameters related to operation of the blood pump 100.

Once values of parameters are calculated or determined at the state observer 177 and/or other locations of the controller 146, some or all of the values of the parameters may be provided to a storage component 179 and/or a hemolysis analyzer 181. The storage component 179 may be and/or may include the memory 154 and/or other suitable memory. Although the storage component 179 is depicted as being part of the controller 146 the storage component 179 may be physically separate from other portions of the controller 146, but this is not required.

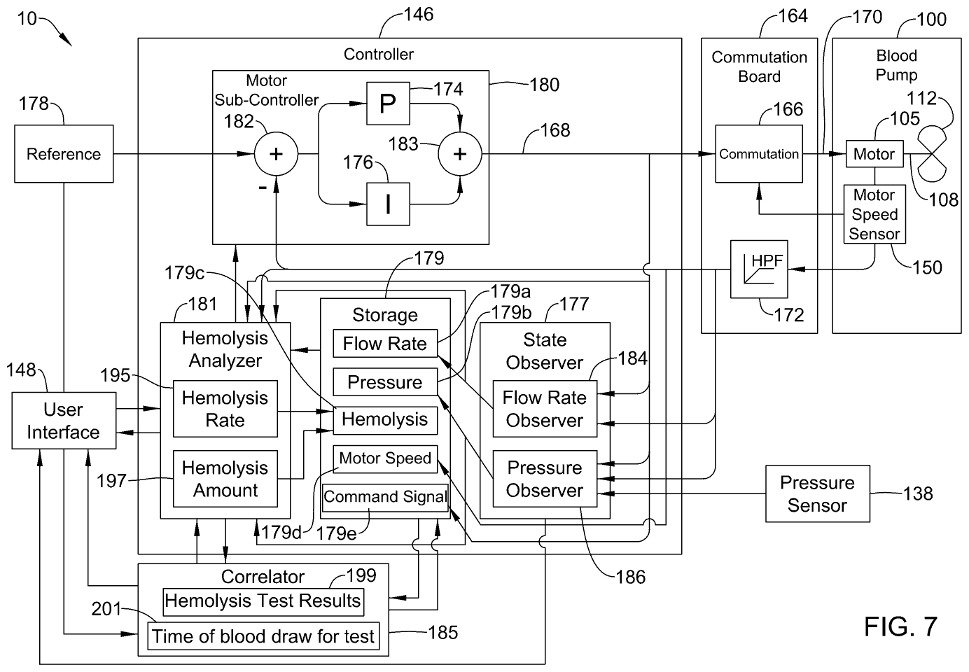

The storage component 179 may be configured in any suitable manner. In some examples, the storage component 179 may be or may include a sample-by-sample storage system, where each value received is individually stored in memory. Additionally or alternatively, in some examples, the storage component 179 may be or may include a filter (e.g., a low pass filter, a high pass filter with a long time constant, etc.) configured to allow for collection of enough calculated or determined values for parameters to analyze the values and detect trends in hemolysis. In some cases, the storage component 179 may be configured to store values received of or from the command signal 168, the motor sensor 150 (e.g., optionally through the HPF 172), the state observer 177, the hemolysis analyzer 181, and/or values of other parameters sensed or determined within the system 10 (e.g., values of motor speed 179d and values of voltage in the command signal 179e, as depicted in FIG. 7, and/or other suitable values of parameters), but this is not required. Further, the storage component 179 may output data to a correlator component 185, the hemolysis analyzer 181, the user interface 148, the motor sub-controller 180, and/or other suitable components of the system 10.

The hemolysis analyzer 181 of the controller 146, which may be in communication with the motor sub-controller 180 and/or the user interface 148, as depicted in FIG. 6, may be configured to analyze data from the state observer 177, data stored in the storage component 179, and/or other suitable data. In some examples, the hemolysis analyzer 181 may be configured to receive information and/or data from one or more sensors, the motor sub-controller 180, the user interface 148, the state observer 177, the storage component 179, a correlator 185, and/or other suitable sources. Example information and/or data received at the hemolysis analyzer 181 may include, but is not limited to, motor speed (e.g., from the sensor 150), vasculature pressure (e.g., from the pressure sensor 138), the command signal 168, the value of the reference parameter 178, hemolysis test result data (e.g., via the user interface 148), times when a blood sample associated with hemolysis test result data was taken, and/or other suitable information and/or data.

The hemolysis analyzer 181 may be configured to output information and/or data to the motor sub-controller 180, the user interface 148, the storage component 179, and/or other component of or in communication with the system 10. In some examples, the hemolysis analyzer 181 may be configured to output a hemolysis rate, a total hemolysis over a time period, hemolysis trends, hemolysis alerts or warnings relative to thresholds, hemolysis test recommendations, control signals (e.g., set points, etc.), hemolysis related pump recommendations, and/or other suitable information, data, and/or control signals. The hemolysis analyzer 181, in some cases, may be configured to determine a rate of hemolysis during operation of the blood pump 100 based on a speed of the motor 105 and/or a voltage value from the command signal 168, but this is not required.

The hemolysis analyzer 181 may utilize any suitable statistical analysis (e.g., linear analysis and/or other suitable statistical analysis) to determine or predict a hemolysis rate and/or amount based on data from the state observer 177 and/or other data. In some examples, the hemolysis analyzer 181 may be configured to process the data received into output using a lookup table (e.g., a multi-dimensional lookup table and/or other suitable type of look up table), a feedback loop, a model, an algorithm, and/or via other suitable techniques.

The correlator component 185 of the controller 146 may be configured to receive hemolysis measurement or test data through manual entry via the user interface and/or communication with systems performing the hemolysis tests. In some examples, the hemolysis measurement or test data may include, but is not limited to, hemolysis blood test results, a time a blood sample was taken from a patient for use in the hemolysis blood tests, and/or other suitable hemolysis measurement data. In operation, the correlator component 185 may utilize the received hemolysis measurement data and data from the state observer (e.g., either directly or via the storage component 179) from a time a blood sample was taken for use in the hemolysis blood test for which results are received and determine a new and/or updated set of coefficients (e.g., lookup table coefficients, polynomial coefficients, etc.) for use by the hemolysis analyzer 181 in determining a hemolysis rate and/or amount based on outputs of the state observer 177. The new and/or updated set of coefficients may be outputted to the hemolysis analyzer 181 and/or other component(s) of or in communication with the controller 146.

FIG. 7 is a schematic view of the system 10, with additional structure of the state observer 177, the storage component 179, hemolysis analyzer 181, and the correlator 185. Although the state observer 177, the storage component 179, hemolysis analyzer 181, and the correlator 185 are depicted in FIG. 7 as being components in the controller 146, the one or more functions of the state observer 177, the storage component 179, hemolysis analyzer 181, and the correlator 185 may be incorporated into the controller 146 separate from one or more of the state observer 177, the storage component 179, hemolysis analyzer 181, and the correlator 185 depicted in FIG. 7. As such, the state observer 177, the storage component 179, hemolysis analyzer 181, and the correlator 185 and/or one or more components of the state observer 177, the storage component 179, hemolysis analyzer 181, and the correlator 185 may be omitted and the controller 146 may continue to perform the functions of the state observer 177, the storage component 179, hemolysis analyzer 181, and the correlator 185 and/or the components thereof. Additionally or alternatively, one or more of or one or more portions of the state observer 177, the storage component 179, hemolysis analyzer 181, and the correlator 185 and/or one or more components of the state observer 177, the storage component 179, hemolysis analyzer 181, and the correlator 185 may be separate from the controller 146 (e.g., on a same or different computing device) and in communication with the controller 146 (e.g., via a high speed and/or reliable communication platform, which may include a wired and/or wireless connection).

As depicted in FIG. 7, the state observer 177 may include a flow rate observer 184 (e.g., a sub-controller or other suitable observer) configured to calculate or determine one or more values of a flow rate of blood across the blood pump 100 and a pressure observer 186 (e.g., a sub-controller or other suitable observer) configured to calculate or determine one or more values of a left ventricular pressure and/or other suitable pressure values. The flow rate data from the flow rate observer 184, pressure data from the pressure observer 186, and/or calculations based on the flow rate and/or pressure data may be utilized to control operation of the blood pump 100 and/or for other suitable purposes separate from being used with the hemolysis analyzer 181. The state observer 177 may include other suitable observers of or in communication with the controller 146 to calculate or determine values of parameters related to operation of the blood pump 100.

The flow rate observer 184 may be configured to calculate or determine a flow rate of blood across the blood pump 100 based, entirely or at least in part, on values of or in the command signal 168, the sensed motor speed, and/or other data or information relevant to the operation of the blood pump 100. Additionally or alternatively, a flow rate may be sensed directly and filtered or calibrated as necessary. The calculated or determined flow rate may then be stored in a flow rate compartment 179a of the storage component 179.

The pressure observer 186 may be configured to receive flow rate data and/or determine flow rates of blood through the blood pump 100. In some examples, one or more pressure sensors may be part of or in communication with the system 10 and communicate sensed values to the controller 146 and the pressure observer 186, where the pressure sensors may be configured to sense measures of or related to pressure in the left ventricle, differential pressure across the blood pump 100 (e.g., differential pressure between the left ventricle 16 and aorta 20), pressure in the vasculature (e.g., via the pressure sensor 138 sensing a measure related to pressure in the aorta 20), and/or other suitable pressure related to operation of the blood pump 100. Additionally or alternatively, in some examples the pressure observer 186 may be configured to determine or calculate the pressure in the ventricle, the differential pressure across the blood pump 100, the pressure in the vasculature, and/or the suitable pressure related to operation of the blood pump 100 based on data and/or signals received from one or more components of or in communication with the system 10 including, but not limited to, values of or in the command signal 168, the sensed motor speed, values from the pressure sensor 138, and/or other data or information relevant to the operation of the blood pump 100. The calculated or determined pressure may then be stored in a pressure compartment 179b of the storage component 179.

The flow rate and/or the pressure at or proximate the blood pump 100 may be calculated and/or determined in any suitable manner. In some examples, the flow rate and/or the pressure at or proximate the blood pump 100 may be based on Bernoulli's principle:

$$P + \frac{1}{2} 2 ρ {v}^{2} + ρ gh = C \qquad ( 1 )$$

where ρ is fluid density, g is acceleration of the fluid due to gravity, P is a pressure at a point in the fluid, v is a velocity of the fluid at the point, h is a height at the point, and C is a constant based on the physical properties of the working fluid.

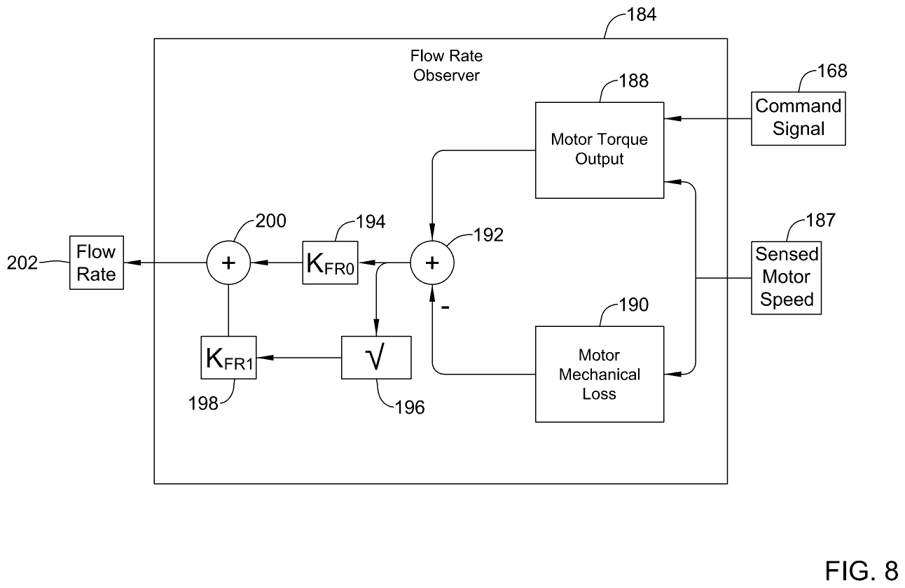

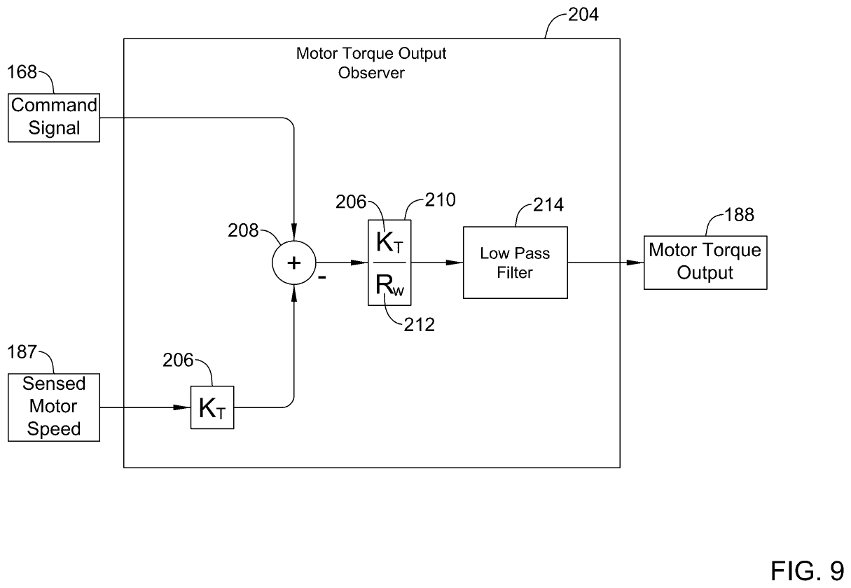

The flow and/or pressure at or proximate the blood pump 100 may be calculated using Newtonian force balance equations; in particular:

$$F = ma \qquad ( 2 )$$

where F is the force imparted by the motor, m is the mass of the fluid in motion, and a is the acceleration imparted upon the working fluid. In some examples, a flow rate may be calculated or determined based on the following function:

$$Flow Rate = f ( {T}_{M} ( {C}_{S} , {S}_{M} ) - {L}_{M} ( {S}_{M} ) ) \qquad ( 3 )$$

where, T_{M} is motor torque, L_{M} is motor mechanical loss, C_{S} is a value of a command signal (e.g., a voltage value or level sent to the motor 105), and S_{M} is a sensed motor speed. An illustrative configuration for calculating or determining a flow rate of blood across the blood pump 100 is described in greater detail with respect to FIGS. 8-10, discussed herein. In some examples, a pressure (e.g., a distal or left ventricular pressure) may be calculated or determined based on the following function:

$$Pressure = f ( ( {P}_{L} ( {T}_{M} ( {C}_{S} , {S}_{M} ) - {L}_{M} ( {S}_{M} ) ) - {P}_{St} ( {S}_{M} ) ) - {P}_{Se} ) \qquad ( 3 )$$

where, P_{L} is pressure loss, P_{St} is stall pressure, and P_{Se} is sensed pressure (e.g., via the pressure sensor 138). An illustrative configuration for calculating or determining a left ventricular pressure is described in greater detail with respect to FIGS. 9-13, discussed herein.

The hemolysis analyzer 181 may be configured to utilize values (e.g., one or more values, two or more values over time, etc.) of one or more of calculated or determined flow rate and pressure, and/or other suitable data to calculate or determine a hemolysis rate and/or hemolysis amount. As depicted in FIG. 7, the hemolysis analyzer 181 may include a hemolysis rate component 195 and a hemolysis amount component 197, among other suitable components. Although the hemolysis rate component 195 and the hemolysis amount component 197 are depicted in FIG. 7 as being part of the hemolysis analyzer 181, one or more of the components may be separate from the hemolysis analyzer 181.

The hemolysis rate component 195 and/or the hemolysis amount component 197 may determine the hemolysis rate and/or the hemolysis amount using one or more lookup tables (e.g., multidimensional lookup tables, algorithms, etc.) that include coefficients, which may be updated over time. The calculated or determined hemolysis rate and/or hemolysis amount may then be stored in a hemolysis compartment 179c of the storage component 179, provided to the user interface 148 for display and/or analysis, and/or to the motor sub-controller 180 for use in controlling operation of the blood pump.

The hemolysis rate component 195 may be configured to calculate or determine a current hemolysis rate due to operation of the blood pump 100 in the patient, provide trend analysis of hemolysis rates over time, provide results of comparing hemolysis rates to thresholds, make determinations based on hemolysis rates, and/or make other hemolysis related determinations. A hemolysis rate may be or may be indicative of a number of red blood cells that are ruptured over a unit of time (e.g., a second, a minute, an hour, etc.) or rotation of the impeller 112.

The hemolysis rate component 195 may be configured to determine a current hemolysis rate due to operation of the blood pump 100 utilizing a hemolysis rate algorithm(s), lookup table(s), or function(s). In some examples, the hemolysis rate algorithm, lookup table, or function may be configured to determine a hemolysis rate based on one or more factors including, but not limited to, hemolysis test results (e.g., a measure of hemolysis in a blood sample and a time at which the blood sample was taken from a patient) on patient blood samples, TR_{H}, a value (e.g., a voltage value) from the command signal 168, C_{S}, a speed of the motor 105, S_{M}, a vasculature pressure sensed by the pressure sensor 138, P_{Va}, a ventricular pressure, P_{Ve}, a differential pressure across the blood pump 100, P_{D}, a flow rate of blood through the blood pump, FR_{BP}, and/or other suitable factors. In some examples, the hemolysis rate may be determined based on the following algorithm:

$$Hemolysis Rate = f ( {C}_{S} , {TR}_{H} , {S}_{M} , {P}_{Va} , {P}_{Ve} , {P}_{D} , {FR}_{BP} ) \qquad ( 4 )$$

In one example, the hemolysis rate may be calculated based on a determined or calculated measure related to flow rate across the blood pump 100 (e.g., a value of a flow rate) and a determined or calculated measure related to left ventricular pressure (e.g., a value of left ventricular pressure), where the measure related to the flow rate across the blood pump 100 and the measured related to the left ventricular pressure may be determined or calculated based on a measure related to the speed of the motor (e.g., the command signal 168, the sensed speed of the motor, etc.) and/or other suitable parameters.

The hemolysis amount component 197 may be configured to calculate an amount of hemolysis over a specified time period based on the determined hemolysis rate. In some examples, the hemolysis amount component 197 may be configured to integrate the hemolysis rate over a specified time period to obtain a total hemolysis over the specified time period. The specified time period may be included, but is not limited to, a time period set by a user via the user interface, a total time period the blood pump has been operating, and/or other suitable time period. In one example, the hemolysis amount component 197 may be configured to integrate the hemolysis rate for at least an instantaneous flow rate of fluid through the blood pump 100 and the ventricular pressure over an entire time the pump is running, but this is not required. Further, when the hemolysis analyzer 181 receives hemolysis test results for a blood sample taken prior to a current time, the hemolysis amount component 197 may be configured to update an amount of hemolysis since the time the sample was taken in view of incorporating the hemolysis test results into the hemolysis rate algorithm.

Although not required, the hemolysis analyzer 181 of the controller 146 may be configured to analyze determined or calculated hemolysis rates and/or hemolysis amounts and provide outputs to the user interface 148, the motor sub-controller 180, the correlator 185, and/or components of or in communication with the system 10. In some cases, the hemolysis analyzer 181 may compare determined or calculated hemolysis rates, hemolysis amounts, and/or values (e.g., averages, rolling averages, median values, etc.) based on the hemolysis rates and/or hemolysis amounts to one or more thresholds and provide outputs based on the comparison. The thresholds may be preset and/or adjustable by a physician or clinician based on the patient's needs, reactions to use of the blood pump 100, and/or other factors. The outputs based on the comparison may be sent to the motor sub-controller 180, the user interface 148, one or more remote patient monitoring systems, and/or other components in communication with the controller 146 of the system 10.

The hemolysis analyzer 181 may be configured to compare determined or calculated hemolysis rates, hemolysis amounts, and/or values (e.g., averages, rolling averages, median values, etc.) based on the hemolysis rates and/or hemolysis amounts to one or more warning thresholds and output signals based on the comparison to the user interface 148 that may trigger the user interface to provide one or more alerts based on the signals received from the hemolysis analyzer 181. In some examples, the warning thresholds may include one or more thresholds at which a physician or clinician may want to take an action or at least be aware of a level of the hemolysis rate and/or hemolysis amount. In some cases, there may be two more warning thresholds with increasing level of severity, which may be indicated at the user interface 148 using increasingly obtrusive warning signals (e.g., lights, sound, tactile feedback, etc.) associated with the thresholds of increasing levels of severity, but this is not required.

The hemolysis analyzer 181 may be configured to compare the calculated or determined hemolysis rate and/or the hemolysis amount to a hemolysis testing threshold at which the hemolysis analyzer 181 may output a signal to the user interface 148 and/or other component in communication with the controller 146 indicating a hemolysis test is recommended to determine an actual hemolysis of the patient proximate the blood pump 100. In one example, the hemolysis analyzer 181 may output a signal indicating a hemolysis test is recommended when an amount of hemolysis during operation of the pump has reached or gone beyond the hemolysis testing threshold. Utilizing a hemolysis testing threshold may facilitate obtaining an actual hemolysis count and taking needed action to avoid adverse effects of hemolysis prior to identifying or observing any adverse effects in the patient.

The hemolysis testing threshold may be a threshold of or associated with the warning thresholds. In some examples, the hemolysis testing threshold may be a middle threshold of three warning thresholds, but this is not required and the hemolysis testing threshold may be a separate threshold from the warning thresholds.

The hemolysis testing threshold and/or other thresholds used by the hemolysis analyzer 181 may be dynamic. In some examples, the hemolysis testing threshold may automatically change over time, such that the hemolysis testing threshold may be higher at a time closer to the latest sample collection time associated with a hemolysis test result and lower at a time further away from the latest sample collection time associated with a hemolysis test result.

The hemolysis analyzer 181 may be configured to compare the calculated or determined hemolysis rate and/or the hemolysis amount to one or more blood pump escalation thresholds. The blood pump escalation threshold may be a threshold of or associated with the warning thresholds, but this is not required. Other dynamic thresholds are contemplated.

Comparing the calculated or determined hemolysis rate and/or the hemolysis amount to the device escalation threshold may result in the hemolysis analyzer 181 outputting an indication that a blood pump 100 having different characteristics (e.g., size of impeller, flow rate capability, etc.) is recommended for use with the patient to reduce hemolysis as the blood of the patient is pumped by the blood pump 100. For example, a physician may increase a speed of the motor 105 of the blood pump 100 to increase a flow rate across the blood pump 100, but doing so may increase the calculated or determined hemolysis rate or expected amount of hemolysis moving forward above a threshold of the implanted blood pump 100, such that the hemolysis analyzer 181 may recommend a more powerful blood pump 100 be positioned within the patient that can pump at higher rates without causing as much hemolysis (e.g., a larger diameter impeller 112 configured to pump more blood at lower rates of rotation, etc.).

The hemolysis analyzer 181 may be configured to compare the calculated or determined hemolysis rate and/or the hemolysis amount to one or more control thresholds. The control thresholds may be thresholds of or associated with the warning thresholds such that when a warning threshold having an associated control threshold is reached or exceeded the hemolysis analyzer 181 may output a signal to the motor sub-controller 180 (e.g., the summer 182) to adjust operation (e.g., increase rotation or decrease rotation) of the motor 105, but this is not required. In some examples, the controller 146 may automatically adjust the command signal 168 based on the calculated or determined hemolysis rate and/or the hemolysis amount during the operation of the blood pump 100. When the hemolysis rate and/or amount is above a threshold, the controller 146 may automatically adjust the command signal 168 to reduce a flow rate of blood through the blood pump 100.

Comparing the calculated or determined hemolysis rate and/or the hemolysis amount to the control thresholds may result in automated control of the motor 105 of the blood pump 100 to mitigate the likelihood of adverse effects manifesting as a result of blood-pump-caused hemolysis. In some cases, when the calculated or determined hemolysis rate and/or the hemolysis amount reaches or goes beyond each of one or more control thresholds, the hemolysis analyzer 181 may output a signal to the motor sub-controller 180 and/or the user interface 148 that adjusts the value of the reference parameter 178 such that the motor 105 and the impeller 112 are adjusted to achieve a desired hemolysis rate and/or hemolysis amount over time or per unit time.

The hemolysis analyzer 181 may be configured to receive the value of the reference parameter 178 inputted at the user interface 148 or a value related to a value of the reference parameter 178 (e.g., a speed of the motor or a value related to the speed of the motor) (e.g., via communication with the user interface 148). The hemolysis analyzer 181 may utilize the value of the reference parameter 178 received to determine or calculate an expected rate of hemolysis or an expected amount of hemolysis during a period of time and compare the expected rate of hemolysis or expected amount of hemolysis to one or more of the thresholds discussed herein or otherwise. In one example, an expected hemolysis rate or expected hemolysis amount determined or calculated based on the value of the reference parameter 178 may be compared to a device escalation threshold and if the determined or calculated expected hemolysis rate or expected hemolysis amount reaches or exceeds the device escalation threshold, the hemolysis analyzer 181 may output an indication that a blood pump change is recommended.

The correlator 185 may be configured to be populated (e.g., manually or automatically from a testing system) with and/or be configured to analyze hemolysis test result data, where the hemolysis test result data may provide a measure of an amount of hemolysis that is occurring in a patient proximate the blood pump 100. The hemolysis test result data may be stored in a hemolysis test results component 199 and times that blood samples used in the hemolysis tests were taken from the patient may be stored in a time of blood draw for testing component 201, but the data may be stored at other suitable locations. The test results related to hemolysis may be manually inputted by a user via the user interface 148 and/or automatically fed to the controller 146 from one or more testing components and/or databases in communication with the controller 146.

The hemolysis test results for a particular patient may be utilized for the calculation of the hemolysis rate in any suitable manner. Although a hemolysis rate algorithm may be set for a population, the hemolysis rate algorithm may be tuned, calibrated, or correlated to a particular patient by the correlator 185 utilizing results of one or more hemolysis tests on one or more patient blood samples obtained over time (e.g., measurements of hemolysis in a blood sample and a time at which the sample was taken), medication information for the patient, information obtained from an IV pump, and/or information obtained from a user and/or one or more other suitable devices or systems in communication with the user interface 148, the controller 146, and/or other component of the system 10. In some examples, the hemolysis test result data, the state observer data obtained or calculated at or proximate a time a blood sample tested was taken from a patient, and/or other suitable data may be utilized as a variable parameter, such that a variable of the hemolysis rate algorithm may change as hemolysis test results are received at the controller 146. Additionally or alternatively, the correlator 185 may be configured to utilize the time organized hemolysis test result data, the state observer data obtained or calculated at or proximate a time a blood sample tested was taken from a patient, and/or other suitable data to determine and/or update coefficients for the lookup table and/or algorithm used by the hemolysis analyzer 181 to calculate or determine hemolysis rates and/or hemolysis amounts (e.g., over a specified period of time). In some examples, the hemolysis test results may be utilized to calculate coefficients for one or more variables (e.g., one or more of: a value (e.g., a voltage value) from the command signal 168, C_{S}, a speed of the motor 105, S_{M}, a vasculature pressure sensed by the pressure sensor 138, P_{Va}, a ventricular pressure, P_{Ve}, a differential pressure across the blood pump 100, P_{D}, a flow rate of blood through the blood pump, FR_{BP}, and/or other suitable variables) utilized in the hemolysis rate algorithm. Once determined, the coefficients may be provided to the hemolysis analyzer 181 and/or the storage component 179.

Any suitable test may be utilized to determine a measured level of hemolysis at a specified time, where suitable tests may check for low red blood cell counts. Example suitable tests producing values of or for use in determining a measured level of hemolysis include, but are not limited to, serum free hemoglobin tests, complete blood count (CBC) tests, reticulocyte count tests, peripheral blood smear tests, lactate dehydrogenase (LDH) tests, a measure of plasma-free hemoglobin in a blood sample, haptoglobin tests, direct Combs test, urinalysis tests, and/or other suitable tests.

The hemolysis test results may be utilized for treating the patient and/or controlling the operation of the blood pump 100. For example, the hemolysis test results may be utilized in a hemolysis trend analysis, to determine control operations for operating the blood pump 100, to update coefficients of an algorithm or lookup table for calculating or determining a hemolysis rate based on flow rate of blood through the blood pump 100 and/or pressure proximate the blood pump 100, etc. In one example, a measured value of hemolysis identified during a hemolysis test and a time at which a sample was taken from a patient for the test may be inputted into a database at the correlator 185 and used to develop, calibrate, and/or update an algorithm for determining or calculating a hemolysis rate. Using both the measured value of hemolysis and the time at which a sample was taken from a patient in developing, calibrating, and/or updating the algorithm for determining or calculating the rate of hemolysis may allow for more accurate hemolysis determinations or calculations at a current time relative to when the algorithm is not receiving updated hemolysis test results overtime as a patient's hemolysis can change over time and the change is not necessarily linearly proportional to the speed of the motor 105 or time since the last test.

FIGS. 8-13 depict schematic view of illustrative controller configurations for calculating or determining values of parameters. Example suitable techniques for calculating or determining values of parameters are described in U.S. Patent Application No. 63/540,346, filed on Sep. 25, 2023, entitled CIRCULATORY SUPPORT DEVICES, SYSTEM, AND METHODS, which is hereby incorporated by reference in its entirety for any and all purposes.

The flow rate observer 184 and the pressure observer 186 may utilize the command signal 168 output from the motor sub-controller 180 as a parameter value representing an operation of the motor 105. In some cases, the command signal 168 may include a voltage level or value or voltage signal configured to achieve a desired motor rotation or speed. The voltage level or value in the command signal 168 may be utilized to determine parameter values rather than using a sensed current at the motor 105 and may provide benefits over the use of such a sensed current at the motor 105 for determining parameter values related to operation of the blood pump 100. For example, using the command signal 168 may allow for faster flow rate and/or pressure determination or calculation times relative to using a value of current sensed at the motor 105 to determine parameter values due to not having to wait until a motor implements the command signal 168 and a sensor senses current used by the motor 105 operating in response to the implemented command signal 168. Use of the command signal 168 as an input to for determining parameter values may allow for determining parameter values based on a view of how the motor 105 will act rather than how the motor 105 was acting, which is identified with a current or voltage sensed at the motor 105, due to the time it takes to move the value of the sensed current or voltage to the controller 146, which may include passing the value of the sensed current or voltage through one or more filters (e.g., the HPF 172 and/or other suitable filters). Additionally, using voltage level(s) or value(s) of the command signal 168 may reduce an amount of noise in the determination of parameter values when compared to noise levels when utilizing sensed values of current and/or voltage at the motor 105 for determining parameter values, which reduces the complexity of determining or calculating the flow rate and/or the pressure.

FIG. 8 schematically depicts a diagram of an illustrative operation of the flow rate observer 184 configured to determine or calculate a flow rate of blood flowing across the blood pump 100. The flow rate observer 184 may be configured to receive a value from the command signal 168 (e.g., a voltage level or value and/or other suitable value) and a sensed motor speed 187 or value related thereto. Based on the received signals or values, the flow rate observer 184 may determine or calculate a motor torque output 188 for the motor 105 and a mechanical loss 190 (e.g., an amount of torque or energy needed to cause the motor 105 to rotate) for the motor 105.

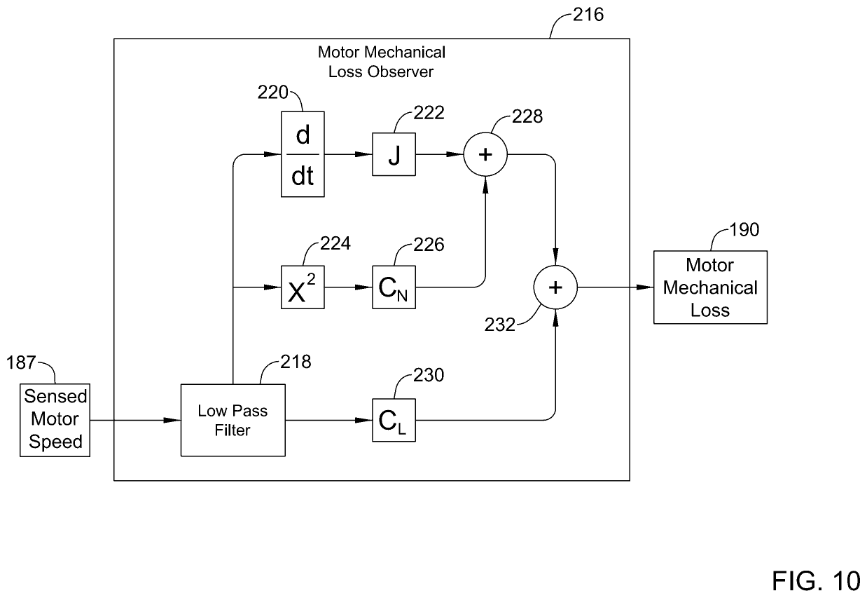

The motor torque output 188 and the mechanical loss 190 may be determined in any suitable manners. An illustrative configuration for determining motor torque output 188 is discussed with respect to FIG. 9. An illustrative configuration for determining mechanical loss 190 is discussed with respect to FIG. 10.

Once the motor torque output 188 and the mechanical loss 190 of the motor 105 are determined or calculated, one or more values of the determined motor torque output 188 and one or more values of the mechanical loss 190 of the motor 105 may be summed at the summer 192. In some examples, a difference between the values may be identified at the summer 192. The difference between the motor torque output 188 (e.g., total torque produced by the motor 105) and the mechanical loss 190 of the motor 105 (e.g., the amount of torque needed to rotate the motor 105) represents an amount of torque that is used by the motor 105 to pump blood through the blood pump 100.

Once the difference between the motor torque output 188 and the mechanical loss 190 is identified, one or more pump coefficients (e.g., torque-flow rate pump coefficients) may be applied to the difference in order to relate the determined torque of the motor 105 available for pumping fluid through the blood pump 100 to flow rate of fluid through the blood pump 100. In some examples, the one or more coefficients may be experimentally determined and specific to a configuration of the blood pump 100.

In the example configuration depicted in FIG. 8, two pump coefficients may be separately applied to a value related to the determined difference between the motor torque output 188 and the mechanical loss 190 of the motor 105. A first pump coefficient K_{FR0} 194 may be applied to the difference between the motor torque output 188 and the mechanical loss 190. The first pump coefficient K_{FR0} 194 may be an experimentally developed value for the blood pump 100 (e.g., a value determined for a configuration of the blood pump 100) that correlates motor torque of the blood pump 100 to flow rate through the blood pump 100. Additionally, a square root 196 of the difference between the motor torque output 188 and the mechanical loss 190 may be determined and a second torque-flow rate coefficient K_{FR1} 198 may be applied to a value of the square root 196. The second torque-flow rate coefficient K_{FR1} 198 may be an experimentally developed value for the blood pump 100 that correlates a square root of the motor torque of the blood pump 100 to flow rate through the blood pump 100.

At summer 200, a value determined by applying the first torque-flow rate coefficient K_{FR0} 194 to the difference between the motor torque output 188 and the mechanical loss 190 is summed with a value determined by applying the second torque-flow rate coefficient K_{FR1} 198 to the value of the square root of the difference between the motor torque output 188 and the mechanical loss 190. The sum may be a value of or may be a value related to a determined or calculated flow rate 202 through the blood pump 100. The flow rate 202 may be output to the user interface 148 or other user interface for use by practitioners in treating the patient with the blood pump 100 and/or to automatically control the operation of the blood pump 100 by providing the flow rate determined or calculated to the motor sub-controller 180 and/or in one or more other suitable manners.

FIG. 9 schematically depicts a diagram of an illustrative operation of the motor torque output observer 204 (e.g., a sub-controller or other suitable observer) configured to determine or calculate the motor torque 188 of the motor 105 of the blood pump 100. The motor torque output observer 204 may be configured to receive a value from the command signal 168 (e.g., a voltage value and/or other suitable value) and a sensed motor speed 187 or value related thereto. Based on the received signals or values, the motor torque output observer 204 may determine or calculate the motor torque output 188 for the motor 105.

Once the command signal 168 and the sensed motor speed 187 are received, one or more coefficients may be applied to the values of or related to the command signal 168 and the sensed motor speed 187. In some examples, the one or more coefficients may be experimentally determined and/or specific to a configuration of the motor 105 and correlate a voltage value to torque output for the motor 105. In some cases, one or more of the coefficients relating voltage to torque output may be provided on a data sheet for the motor 105.

In the example configuration depicted in FIG. 9, two coefficients may be separately applied to the received voltage command signal 168 and the sensed motor speed 187, respectively. A torque-voltage coefficient K_{T} 206 may be applied to a value of or a value related to the sensed motor speed 187 to produce a voltage value (e.g., a value of a back EMF of the motor 105 and/or other suitable value) that can be summed with a value of the command signal 168 at the summer 208. The torque-voltage coefficient K_{T} 206 may be the motor torque constant, which may be a parameter value found on a motor data sheet.

At the summer 208, a difference between the value determined by applying the torque-voltage coefficient K_{T} 206 to the sensed motor speed 187 and the value of the command signal 168 may be determined. The value determined by applying the torque-voltage coefficient K_{T} 206 to the sensed motor speed 187 may be the amount of or may be representative of the amount of voltage produce internally to the motor 105 based on the speed of the motor 105. To determine an accurate value of motor torque output 188, the value determined by applying the torque-voltage coefficient K_{T} 206 to the sensed motor speed 187 may be subtracted from the commanded voltage in the command signal 168.

A ratio coefficient 210 may be applied to the difference between the value determined by applying the torque-voltage coefficient K_{T} 206 to the sensed motor speed 187 and the value of the command signal 168. The ratio coefficient 210 may be determined by dividing the torque voltage coefficient K_{T} 206 by a winding resistance of the motor 105. The winding (or terminal) resistance R_{w} 212 of the motor 105 may be experimentally determined for the motor 105 and/or the winding resistance R_{w} 212 of the motor 105 may be a value found on a data sheet of the motor 105.

The output of the ratio coefficient 210 applied to the difference between the value determined by applying the torque-voltage coefficient K_{T} 206 to the sensed motor speed 187 and the value of the command signal 168 may be representative of the motor torque output 188. In some cases, for example as depicted in FIG. 9, a low pass filter 214 may be applied to the value of the output from applying the ratio coefficient 210 to the difference between the value determined by applying the torque-voltage coefficient K_{T} 206 to the sensed motor speed 187 and the value of the command signal 168. The low pass filter 214 may be configured to filter out all values that have a higher frequency than a predetermined frequency threshold to filter out noise.

FIG. 10 schematically depicts a diagram of an illustrative operation of a motor mechanical loss observer 216 (e.g., a sub-controller or other suitable observer) configured to determine or calculate the mechanical loss 190 of the motor 105 of the blood pump 100 based on a force balance equation. The motor mechanical loss observer 216 may be configured to receive a sensed motor speed 187 or value related thereto. Based on the received signals or values, the motor mechanical loss observer 216 may determine or calculate the motor mechanical loss 190 for the motor 105.

Once the value(s) of the sensed motor speed 187 are received, a low pass filter 218 may be applied to the value(s) of the sensed motor speed 187. The low pass filter 218 may be the same as the low pass filter 214 with the same frequency threshold or a different low pass filter with a different frequency threshold to filter noise from the value(s) of the sensed motor speed 187 that are received. The output of the low pass filter 218 may be processed in a plurality of separate steps and summed to obtain the determined or calculated motor mechanical loss 190.

In a step, a derivative 220 of the output of low pass filter 218 may provide an acceleration of the motor 105. An inertia equation J 222 may be applied to the determined or calculated acceleration of the motor 105, where the output of applying the inertia equation J 222 to the acceleration of the motor 105 may represent or may be a calculated or determined force required to overcome inertia of the motor 105.

In an additional processing step, the output of the low pass filter 218 may be squared 224 and a non-linear drag coefficient CN 226 may be applied to the square of the output of the low pass filter 218. The non-linear drag coefficient CN 226 may correspond to non-linear forces and/or power lost to the environment as heat due to inefficiencies in the motor 105 and the output of applying the non-linear drag coefficient CN 226 to the output of the low pass filter 218 may represent or may be a calculated or determined force required to overcome non-linear drag on the motor 105.

The calculated or determined force required to overcome inertia of the motor 105 and the calculated or determined force to overcome drag on the motor 105 may be summed at a summer 228. The output of the summer 228 may be, or may be representative of, a calculated or determined net inertia and non-linear force acting on the motor 105.

In a further processing step, a linear drag coefficient CL 230 may be applied to the output of the low pass filter 218. The linear drag coefficient CL 230 may correspond to linear forces and/or power lost to the environment as heat due to inefficiencies in the motor 105 and the output of applying the linear drag coefficient CL 230 to the output of the low pass filter 218 may represent or may be a calculated or determined force required to overcome linear drag on the motor 105.

The calculated or determined net inertia and non-linear force acting on the motor 105 and the calculated or determined force required to overcome linear drag on the motor 105 may be summed at a summer 232. The output of the summer 232 may be or may be representative of a calculated or determined net force (e.g., inertia forces, non-linear drag forces, and linear drag forces) acting on the motor 105. In other words, the output of the summer 232 may be the motor mechanical loss 190.

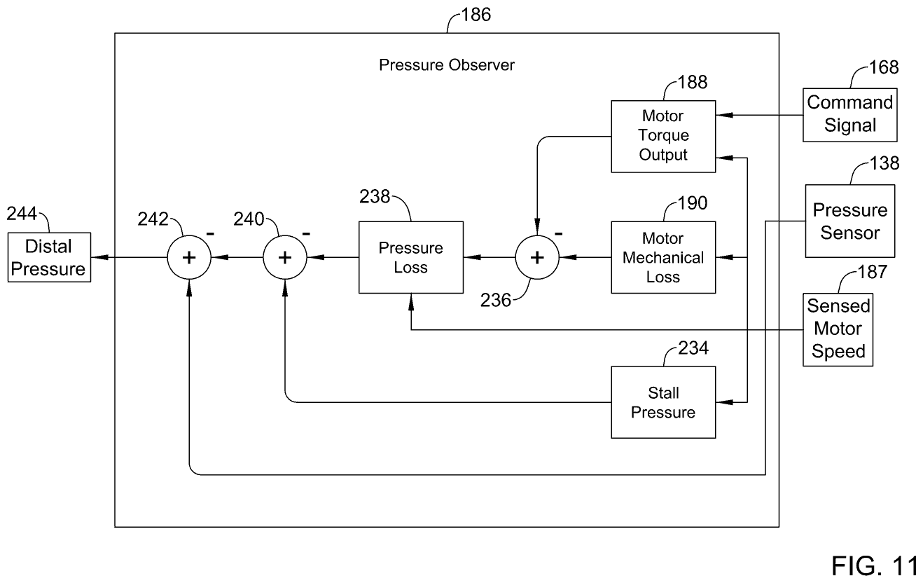

FIG. 11 schematically depicts a diagram of an illustrative operation of the pressure observer 186 configured to determine or calculate a pressure distal of the impeller 112 (or proximal relative to a direction of blood flow) (e.g., a ventricular pressure, such as a left ventricular pressure and/or a right ventricular pressure depending on in which ventricle the blood pump 100 extends), where distal of the impeller may be at a ventricle (e.g., left ventricle or right ventricle of a heart of a patient). The pressure observer 186 may be configured to receive a value from the command signal 168 (e.g., a voltage value or level and/or other suitable value), a sensed motor speed 187 or value related thereto, and a value from the pressure sensor 138 (e.g., a pressure sensor sensing pressure proximal of the impeller 112 or distal relative to a direction of blood flow, such as a pressure in an aorta of a patient). Based on the received signals or values, the pressure observer 186 may determine or calculate a motor torque output 188 for the motor 105, a mechanical loss 190 (e.g., an amount of torque or energy needed to cause the motor 105 to rotate) for the motor 105, and a stall pressure (e.g., a head pressure or a zero flow pressure), which may be a pressure at which blood is no longer moving through the blood pump 100.

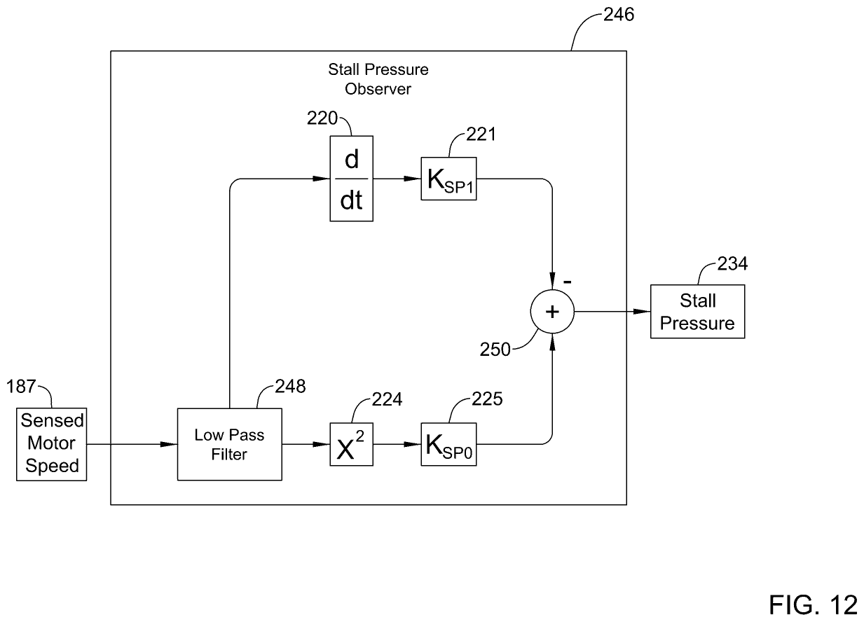

The motor torque output 188 and the mechanical loss 190 may be determined in any suitable manners including, but not limited to, as described herein with respect to FIGS. 9 and 10. Further, the stall pressure 234 may be determined in any suitable manner. An illustrative configuration for determining the stall pressure 234 is discussed with respect to FIG. 12.

Once the motor torque output 188 and the mechanical loss 190 of the motor 105 are determined or calculated, one or more values of the determined motor torque output 188 and one or more values of the mechanical loss 190 of the motor 105 may be summed at the summer 236. In some examples, a difference between the values may be identified at the summer 236, which may represent an amount of torque that is used by the motor 105 to pump blood through the blood pump 100, similar to as discussed above with respect to FIG. 9.

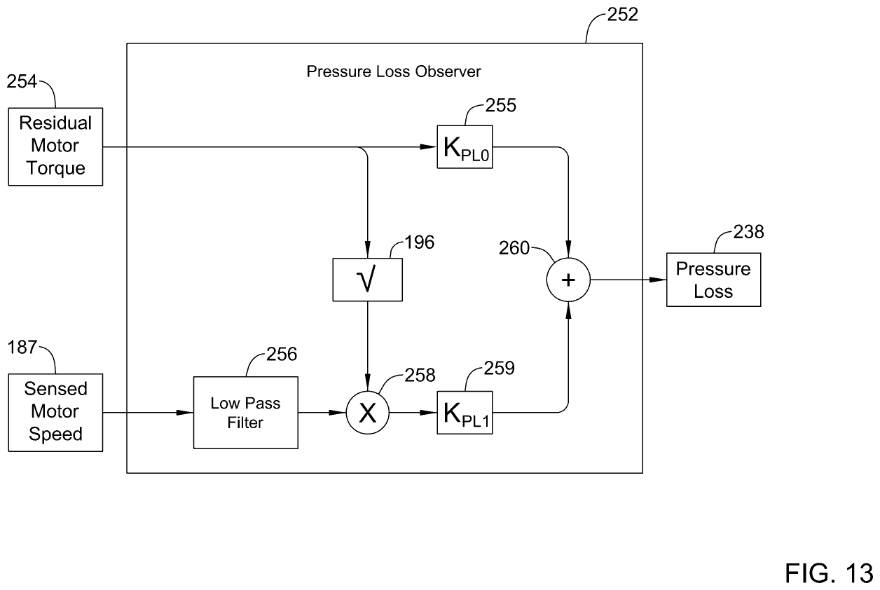

Once the difference between the motor torque output 188 and the mechanical loss 190 is identified, the pressure observer 186 may utilize the difference between the motor torque output 188 and the mechanical loss 190 and the sensed motor speed 187 to calculate or determine a pressure loss 238 caused by blood flow through the blood pump 100. An illustrative configuration for determining the pressure loss 238 is discussed with respect to FIG. 13.

In the example configuration depicted in FIG. 11, the calculated or determined pressure loss 238 may be summed with the calculated or determined stall pressure 234 at a summer 240. A difference between the stall pressure 234 and the pressure loss 238 may be and/or may represent an instantaneous pressure drop across the pump for a given flow of blood through the blood pump 100. In some examples, the pressure drop represents the difference between a ventricular pressure for a ventricle (e.g., the left ventricle or right ventricle) in which the blood pump is positioned and an arterial pressure for an artery in which the blood pump 100 is positioned (e.g., the aorta or left pulmonary artery).

The determined pressure drop across the blood pump 100 may be summed to a pressure value sensed by and/or a pressure value based on a measure sensed by the pressure sensor 138 (e.g., at a pressure at a location proximal of the impeller 112, such as at an aorta of the patient) at a summer 242. At the summer 242, the pressure value may be subtracted from the determined pressure drop across the blood pump 100 to determine a distal pressure value 244 of a pressure distal of the impeller 112 (e.g., a ventricular pressure, such as the left or right ventricular pressure). The distal pressure value 244 may be output to the user interface 148 or other user interface for use by practitioners in treating the patient with the blood pump 100 and/or to automatically control the operation of the blood pump 100 by providing the distal pressure value 244 determined or calculated to the motor sub-controller 180 and/or may be output in one or more other suitable manners.

FIG. 12 schematically depicts a diagram of an illustrative operation of a motor stall pressure observer 246 (e.g., a sub-controller or other suitable observer) configured to determine or calculate the stall pressure 234 of the motor 105 of the blood pump 100. The stall pressure observer 246 may be configured to receive the sensed motor speed 187 or a value related thereto. Based on the received signals or values, the stall pressure observer 246 may determine or calculate the stall pressure 234 for the motor 105.

Once the value(s) of the sensed motor speed 187 are received, a low pass filter 248 may be applied to the value(s) of the sensed motor speed 187. The low pass filter 248 may be the same as one or both of the low pass filter 214, 218 with the same frequency threshold or a different low pass filter with a different frequency threshold to filter noise from the value(s) of the sensed motor speed 187 that are received. The output of the low pass filter 218 may be processed in a plurality of separate steps to obtain the determined or calculated stall pressure 234 of the motor 105.

In a step, a derivative 220 of the output of low pass filter 248 may provide an acceleration of the motor 105. In an additional processing step, the output of the low pass filter 248 may be squared 224.

Once the acceleration of the motor 105 is determined and the output of the low pass filter 248 is squared, one or more coefficients (e.g., a speed-pressure loss pump coefficient) may be applied thereto in order to relate the sensed motor speed 187 to stall pressure 234. In some examples, the one or more pump coefficients may be experimentally determined and specific to a configuration of the blood pump 100. In some examples, a second pump coefficient K_{SP1} 221 may be applied to the acceleration of the motor 105 and a first pump coefficient K_{SP0} 225 may be applied to the squared motor speed output from the low pass filter 248. The second pump coefficient K_{SP1} 221 may be an experimentally developed value for the blood pump 100 (e.g., a value determined for a configuration of the blood pump 100) that correlates an acceleration of the motor 105 of the blood pump 100 to stall pressure of the blood pump 100. The first pump coefficient K_{SP0} 225 may be an experimentally developed value for the blood pump 100 that correlates a square of the sensed motor speed 187 to stall pressure of the blood pump 100.

The output of applying the first pump coefficient K_{SP0} to the output of the low pass filter 248 may be subtracted from the output of applying the second pump coefficient K_{SP1} to the acceleration of the motor 105 at a summer 250. The difference between the output of applying the first pump coefficient K_{SP0} to the squared output of the low pass filter 248 and the output of applying the second pump coefficient K_{SP1} to the acceleration of the motor 105 determined at a summer 250 may be the stall pressure 234, where the stall pressure 234 may be the maximum pressure the blood pump 100 may be able to produce.

FIG. 13 schematically depicts a diagram of an illustrative operation of a pressure loss observer 252 (e.g., a sub-controller or other suitable observer) configured to determine or calculate the pressure loss 238 of the motor 105 of the blood pump 100. The pressure loss observer 252 may be configured to receive the sensed motor speed 187 or a value related thereto and a residual motor torque 254 or values related thereto. The residual motor torque 254 may be a difference between the motor torque output 188 and the motor mechanical loss 190. Based on the received signals or values, the pressure loss observer 252 may determine or calculate the pressure loss 238 for the motor 105.

Once the value(s) of the sensed motor speed 187 and the residual motor torque 254 are received, a low pass filter 256 may be applied to the value(s) of the sensed motor speed 187. The low pass filter 256 may be the same as one or both of the low pass filter 214, 218, 248 with the same frequency threshold or a different low pass filter with a different frequency threshold to filter noise from the value(s) of the sensed motor speed 187 that are received. The output of the low pass filter 218 may be provided to a multiplier 258.

The residual motor torque 254 may be processed in a plurality of steps using one or more coefficients that may relate residual motor torque 254 and/or sensed motor speed 187 to pressure loss 238. In some examples, the one or more of the coefficients may be experimentally determined and specific to a configuration of the blood pump 100.

In one step, a first pump coefficient K_{PL0} 255 may be applied to the residual motor torque. The first pump coefficient K_{PL0} 255 may be an experimentally developed value for the blood pump 100 (e.g., a value determined for a configuration of the blood pump 100) that correlates residual motor torque of the blood pump 100 to pressure loss 238. The result of the application of the first pump coefficient K_{PL0} 255 may be provided to a summer 260.

Further, a square root 196 of the residual motor torque 254 may be calculated or determined and applied to the multiplier 258. The values received at the multiplier 258 may be multiplied.

Once the values received at the multiplier 258 are multiplied a second pump coefficient K_{PL1} 259 may be applied to the product of the values provided to the multiplier 258. The second pump coefficient K_{PL1} 259 may be an experimentally developed value for the blood pump 100 that correlates a product of residual motor torque 254 and sensed motor speed 187 to pressure loss 238. The result of the application of the second pump coefficient K_{PL1} 259 to the product of the values provided to the multiplier 258, may be provided to summer 260.

At the summer 260, the values resulting from applying the first pump coefficient K_{PL0} 194 to the residual motor torque 254 and the values resulting from applying the second pump coefficient K_{PL1} 259 to the value output from the multiplier 258 may be summed to determine or calculate a value of the pressure loss 238. The determined value of the pressure loss 238 may be used in the determination of a distal or ventricular pressure 244 and/or the determination of other suitable parameters.

The constants or coefficients depicted in and discussed with respect to FIGS. 8-13 may be values based on one or more parameters. In some examples, the constants or coefficients in FIGS. 7-12 may be values scheduled based on motor or pump speed, motor or pump temperature, motor or pump power, motor or pump run-time, a combination of values of these parameters and/or values additional or alternative parameters used to compensate for changes in pump performance as the pump operating conditions change.

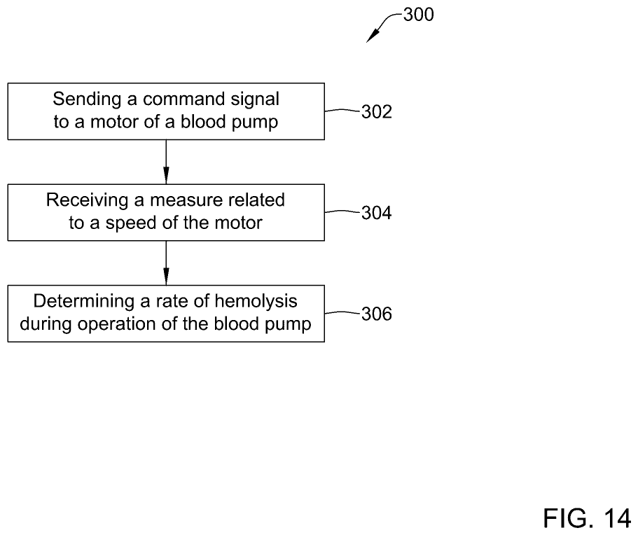

FIG. 14 depicts a schematic method or technique 300 for operating a blood circulatory support system for use with a heart of a patient. The method 300 may include sending 302 a command signal to a motor of a blood pump positioned within a patient. In some examples, the command signal may be sent from a controller to the motor and the command signal may include a voltage value or level for controlling a speed of the motor in a desired manner. In some examples, the command signal may be based on a value of the reference parameter 178 (e.g., a set point) received by a practitioner, the hemolysis analyzer 181, and/or other component of or in communication with the circulatory support system, as discussed herein or otherwise. The command signal may cause the motor to drive a drive component (e.g., an impeller) to pump blood from a ventricle (e.g., a left ventricle or right ventricle) of the patient through the blood pump to arterial vasculature (e.g., an aorta or pulmonary artery) of the patient.

During operation of the blood pump, a speed of the motor may be sensed by one or more sensors sensing a motor speed or a measure related to motor speed. The sensed speed of the motor may be communicated from the one or more sensors sensing the motor speed or a measure related to motor speed to the controller, such that the controller may receive 304 the sensed speed or measure related to speed of the motor and utilize that value in operation of the blood pump.

Further, the method 300 may include determining 306 a rate of hemolysis during operation of the blood pump based on the received value of the speed of or measure related to the speed of the motor. In some examples, the rate of hemolysis during operation of the blood pump may be determined or calculated using only the speed of or measure related to the speed of the motor and/or one or more additional or alternative parameters including, but not limited to, pressure proximate the blood pump, flow through the blood pump, hemolysis test results, and/or other suitable parameters or variables. The determined calculated values of hemolysis rate and/or hemolysis amount may be utilized in value determinations or calculations discussed herein, which may be utilized to control operation the blood pump (e.g., via configuring and/or adjusting command signals from the controller), to assess operations of the blood pump, to treat the patient, and/or to monitor patient needs relative to the blood pump (e.g., determine and/or output an indication of a sufficiency of a blood pump to meet patient needs, such as pumping blood from a left ventricle to an aorta).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A circulatory support system comprising:
a blood pump comprising:
a driven component; and
a motor in communication with the driven component and configured to drive the driven component to pump a blood flow through the blood pump;
one or more sensors configured to sense a measure related to a speed of the motor; and
a controller in communication with the motor and the one or more sensors configured to sense the measure related to the speed of the motor, and
wherein the controller is configured to send a command signal to the motor and determine a rate of hemolysis during operation of the blood pump based on the speed of the motor.

2. The circulatory support system of claim 1, wherein the controller is configured to:
determine a measure related to a flow rate of blood through the blood pump based on the speed of the motor,
determine a measure related to a left ventricular pressure of the patient based on the speed of the motor, and
determine the rate of hemolysis based on the measure related to the flow rate through the blood pump and the measure related to the left ventricular pressure of the patient.

3. The circulatory support system of claim 1, wherein the controller is configured to receive a hemolysis test result for a patient including a time at which a sample of blood was taken from the patient and determine the rate of hemolysis based on the speed of the motor, the hemolysis test result, and the time at which the sample of blood was taken from the patient.

4. The circulatory support system of claim 3, wherein the hemolysis test result includes a value of plasma-free hemoglobin in the sample of blood or a value of lactate dehydrogenase (LDH) in the sample of blood.

5. The circulatory support system of claim 1, wherein the controller is configured to integrate the rate of hemolysis determined during a period of operation of the blood pump to determine an amount of hemolysis that has occurred during the period of operation.

6. The circulatory support system of claim 1, wherein the controller is configured to automatically adjust the command signal based on the rate of hemolysis determined during operation of the blood pump.

7. The circulatory support system of claim 6, wherein the controller is configured to automatically adjust the command signal to reduce a flow rate of blood through the blood pump based on the rate of hemolysis determined during operation of the blood pump.

8. The circulatory support system of claim 1, wherein the controller is configured to:
determine an amount of hemolysis based on the rate of hemolysis determined during operation of the pump, and
output a signal indicating a hemolysis test is recommended when the amount of hemolysis has reached or gone beyond a threshold level.

9. The circulatory support system of claim 1, wherein the controller is configured to:
receive an input from a user;
determine an expected rate of hemolysis during operation of the blood pump based on the input received from the user; and
output an indication that a blood pump change is recommended based on the expected rate of hemolysis determined being above a threshold.

10. The circulatory support system of claim 1, wherein the controller is configured to output an indication to a user interface based on the rate of hemolysis determined during operation of the blood pump.

11. A non-transitory computer readable medium having stored thereon instructions executable by a circulatory support device for use with a heart of a patient, the instructions causing the circulatory support device to perform a method comprising:
sending a command signal from a controller to a motor of a blood pump to cause the motor of the blood pump to drive a driven component to pump blood from a ventricle of the heart of the patient through the blood pump to vasculature of the patient;
receiving a measure related to a speed of the motor from one or more sensors in communication with the controller; and
determining a rate of hemolysis during operation of the blood pump based on the speed of the motor.

12. The non-transitory computer readable medium of claim 11, wherein determining the rate of hemolysis during operation of the blood pump further comprises:
determining a flow rate of blood through the blood pump based on the speed of the motor,
determining a measure related to a ventricular pressure of the patient based on the speed of the motor, and
determining the rate of hemolysis based on the measure related to the flow rate and the measure related to the ventricular pressure of the patient.

13. The non-transitory computer readable medium of claim 11, wherein the method further comprises:
receiving a hemolysis test result at a specified time at which a tested blood sample was taken, and
wherein the rate of hemolysis during operation of the blood pump is determined based on the hemolysis test result and the specified time.

14. The non-transitory computer readable medium of claim 11, wherein the method further comprises:
integrating the rate of hemolysis determined during a period of operation of the blood pump to determine an amount of hemolysis during the period of operation of the blood pump.

15. The non-transitory computer readable medium of claim 14, wherein the method further comprises:
outputting a signal indicating a hemolysis test is recommended when the amount of hemolysis has reached or gone beyond a threshold level.

16. The non-transitory computer readable medium of claim 11, wherein the method further comprises:
automatically adjusting the command signal based on the rate of hemolysis determined during operation of the blood pump.

17. A method of operating a blood circulatory support system for use with a heart of a patient, the method comprising:
sending a command signal from a controller to a motor of a blood pump to cause the motor of the blood pump to drive a driven component to pump blood from a ventricle of the heart of the patient through the blood pump to vasculature of the patient;
receiving a measure related to a speed of the motor from one or more sensors in communication with the controller; and
determining a rate of hemolysis during operation of the blood pump based on the speed of the motor.

18. The method of claim 17, wherein determining the rate of hemolysis during operation of the blood pump further comprises:
determining a flow rate of blood through the blood pump based on the speed of the motor,
determining a measure related to a ventricular pressure of the patient based on the speed of the motor, and
determining the rate of hemolysis based on the measure related to the flow rate and the measure related to the ventricular pressure of the patient.

19. The method of claim 17, further comprising:
determining an amount of hemolysis during a period of operation of the blood pump based on the rate of hemolysis determined during the period of operation of the blood pump; and
outputting a signal indicating a hemolysis test is recommended when the amount of hemolysis has reached or gone beyond a threshold level.

20. The method of claim 17, further comprising:
automatically adjusting the command signal based on the rate of hemolysis determined during operation of the blood pump.
